(19) United States
(12) Patent Application Publication
(10) Pub. No.: US 2024/0299151 A1
(43) Pub. Date: Sep. 12, 2024

(54) EMBOLIC FILTER WITH FLEXION

(71) Applicant: W. L. Gore & Associates, Inc., Newark, DE (US)

(72) Inventor: William D. Montgomery, Flagstaff, AZ (US)

(21) Appl. No.: 18/272,048

(22) PCT Filed: Jan. 14, 2022
(86) PCT No.: PCT/US2022/012571

§ 371 Date: Jul. 12, 2023

Related U.S. Application Data
(60) Provisional application No. 63/137,385, filed on Jan. 14, 2021.

(51) Int. Cl. A61F 2/01

(52) U.S. Cl. CPC A61F 2/0105; A61F 2/011; A61F 2002/016; A61F 2002/018; A61F 2230/0017; A61F 2250/001; A61F 2250/0018

(57) ABSTRACT

Described embodiments are directed toward an embolic filter system. The embolic filter system generally includes an elongate element having a first end and a second end and an embolic filter assembly comprising a frame having a capture section distal to the attachment section, the capture section including a plurality of strut elements arranged to define a plurality of cells having cell ends and at least one flexion element operably coupled to and between cell ends of two adjacent cells of the plurality of cells such that the frame is operable to bend between the cell ends.

### CROSS-REFERENCE TO RELATED APPLICATION

This application is a national phase application of PCT Application No. PCT/US2022/012571, internationally filed on Jan. 14, 2022, which claims the benefit of Provisional Application No. 63/137,385, filed Jan. 14, 2021, which are incorporated herein by reference in their entireties for all purposes.

### INCORPORATION BY REFERENCE

International Patent Application No. PCT/US2019/056737, filed Oct. 17, 2019, which claims benefit to U.S. Provisional Application No. 62/747,026, filed Oct. 17, 2018, the inventors being William Montgomery and Edward Shaw, are incorporated herein by reference in their entireties for all purposes.

### BACKGROUND

Endovascular procedures address a broad array of medical needs, including endovascular access, diagnosis, and/or repair through minimally invasive or relatively less invasive means than surgical approaches. During some endovascular procedures, embolic debris may become dislodged or circulated in the vasculature. Circulation of embolic debris can cause mild to extreme cardiovascular complications, leading to stroke and even death.

Embolic protection devices have been developed and used in connection with such endovascular procedures to help mitigate the risks associated with various endovascular procedures. Some embolic protection devices operate to capture embolic debris and filter the same from the blood. The captured embolic debris can be aspirated (e.g., actively or passively) prior to removal of the embolic protection device. Additionally or alternatively, the embolic protection device can be configured to trap the embolic debris within the embolic protection device such that the embolic debris is retained by the embolic protection device upon its removal from the vasculature. However, a common risk of these procedures is the unintentional release of some or all of the captured embolic debris back into the vasculature during the removal process.

Proper orientation of the embolic protection devices within the vasculature is an important factor in the facilitation of embolic debris capture and removal. However, while conventional devices may be deployable within tortuous vasculature, some lack the means for orienting or repositioning the device within the vasculature after it has been deployed. Poor orientation of embolic protection devices may result in embolic debris bypassing the embolic protection device, such as by way of one or more gaps between the embolic protection device and a vessel wall and/or by embolic debris not being fully captured by the embolic protection device resulting in unintended ejection of the embolic debris back into the blood upon removal of the embolic protection device from the vasculature. Proper orientation is especially difficult in tortuous anatomy.

### SUMMARY

According to a first example (“Example 1”), a medical device includes an elongate element having a first end and a second end, and an embolic filter assembly comprising a frame having a capture section distal to the attachment section, the capture section including a plurality of strut elements arranged to define a plurality of cells having cell ends and at least one flexion element operably coupled to and between cell ends of two adjacent cells of the plurality of cells such that the frame is operable to bend between the cell ends.

According to another example (“Example 2”) further to Example 1, the capture section is radially expandable relative to the attachment section such that the embolic filter assembly is configured to transition from a compressed state toward an expanded state in situ.

According to another example (“Example 3”) further to any of the preceding Examples, the capture section includes a seal establishment zone operable to establish a seal with tissue of a patient and a flexion zone operable to flex to conform to tortious anatomy of a patient.

According to another example (“Example 4”) further to Example 3, the flexion element defines the flexion zone in which the frame has greater flexibility than in adjacent portions of the frame.

According to another example (“Example 5”) further to any of the preceding Examples, the frame is configured such that the capture section is configured to self-expand.

According to another example (“Example 6”) further to any of the preceding Examples, wherein the frame includes a metallic material.

According to another example (“Example 7”) further to Example 6, the metallic material includes nitinol.

According to another example (“Example 8”) further to any of the preceding Examples, wherein the frame is of a unibody construction such that the attachment section and the capture section are formed by a single monolithic component.

According to another example (“Example 9”) further to any of the preceding Examples, wherein the flexion element is serpentine shaped having at least one curve in a first direction and at least one curve in a second, opposite direction from the first direction.

According to another example (“Example 10”) further to any of the preceding Examples, wherein the strut elements have a square, rectangular, trapezoidal, rounded square, rounded rectangular, or rounded trapezoidal shape.

According to another example (“Example 11”) further to Example 10, the cut tube is a laser cut tube.

According to another example (“Example 12”) further to any of Examples 9 to 10, the frame includes an intermediate section that is helical.

According to another example (“Example 13”) further to any of the preceding Examples, the plurality of cells are closed cells that are quadrilateral.

According to another example (“Example 14”) further to Example 13, each of the plurality of closed cells includes a first apex and a second apex opposite the first apex.

According to another example (“Example 15”) further to Example 14, the at least one flexion element is operably coupled between the proximal apex of a first closed cell of the plurality of closed cells and the distal apex of a second closed cell of the plurality of closed cells.

According to another example (“Example 16”) further to Example 14, the plurality of closed cells form a first cell row and a second cell row along a longitudinal length of the frame, wherein each distal apex of the plurality of closed cells of the first cell row is operably coupled to each proximal apex of the plurality of closed cells of the second cells via the flexion element.

According to another example (“Example 17”) further to any of the preceding Examples, the at least one flexion element is adapted to bend at a plurality of flex points between first and second ends of the flexion element.

According to another example (“Example 18”) further to any of the preceding Examples, the at least one flexion element is adapted to extend in length.

According to another example (“Example 19”) further to Example 3, the frame defines a longitudinal axis, wherein the frame is operable to flex at the flexion zone such that the longitudinal axis is nonlinear when the flexion zone of the frame flexes.

According to another example (“Example 20”) further to any preceding Example, wherein the frame is adjustably deployable such that the at least one flexion element is operable to partially mechanically isolate the plurality of closed cells from each other during expansion of the frame element.

According to a second example (“Example 21”), an embolic filter assembly for deployment in a lumen of a patient includes a frame disposed about a longitudinal axis and operable to expand from a smaller, collapsed configuration to a larger, expanded configuration, the frame including a plurality of frame elements defining a sealing section and a capture section, the sealing section configured to interface with the lumen of the patient, the capture section including flexion elements positioned between frame elements within the capture zone, the frame being operable to bend away from the longitudinal axis at the flexion elements more than adjacent portions of the frame without flexion elements.

According to another example (“Example 22”) further to Example 21, the flexion elements are aligned around a circumference of the frame at a common, longitudinal position along a length of the frame.

According to another example (“Example 23”) further to Example 21, the embolic filter assembly further includes a filter element coupled to the frame.

According to another example (“Example 24”) further to Example 21, the frame is operable to be partially deployed such that the sealing section is operable to expanded to an expanded diameter while at least a portion of the capture section is maintained at a collapsed diameter.

According to another example (“Example 25”) further to Example 24, the capture section is operable to filter fluid flowing through the capture section when the capture section is partially deployed.

According to another example (“Example 26”) further to Example 21, the frame is configured to permit delivery of secondary devices.

According to another example (“Example 27”) further to Example 21, the frame is operable to angulate at the flexion elements to maintain the frame in an orthogonal orientation within the lumen when deployed.

According to another example (“Example 28”) further to Example 27, the flexion elements include a curved shape, a bent shape, a zigzag shape, a serpentine shape, or a coil shape.

According to another example (“Example 29”) further to Example 21, the frame elements form a plurality of closed cells, each closed cell including a proximal apex and a distal apex.

According to another example (“Example 30”) further to Example 29, a first flexion element is operably coupled between the proximal apex of a first closed cell of the plurality of closed cells and the distal apex of a second closed cell of the plurality of closed cells.

According to another example (“Example 31”) further to Example 29, the plurality of closed cells form a first cell row and a second cell row along a longitudinal length of the frame, wherein each distal apex of the plurality of closed cells of the first cell row is operably coupled to each proximal apex of the plurality of closed cells of the second cells via the flexion element.

According to another example (“Example 32”) further to Example 29, the frame is adjustably deployable such that the flexion elements are operable to partially mechanically isolate the plurality of closed cells from each other.

According to a third example (“Example 33”), an embolic filter assembly for deployment in a lumen of a patient includes a frame disposed about a longitudinal axis and operable to expand from a smaller, collapsed configuration to a larger, expanded configuration, the frame including a plurality of frame elements defining a sealing section and a capture section, the sealing section configured to interface with the lumen of the patient, the capture section including flexion elements positioned between frame elements within the capture zone, the frame being operable to be partially deployed such that the sealing section is operable to expanded to an expanded diameter while at least a portion of the capture section is maintained at a collapsed diameter.

While multiple examples are disclosed, still other examples will become apparent to those skilled in the art from the following detailed description, which shows and describes illustrative examples. Accordingly, the drawings and detailed description are to be regarded as illustrative in nature and not restrictive.

### BRIEF DESCRIPTION OF THE DRAWINGS

The accompanying drawings are included to provide a further understanding of the disclosure and are incorporated in and constitute a part of this specification, illustrate examples, and together with the description serve to explain the principles of the disclosure.

FIG. 1 is an illustration of an embolic filter frame, according to some embodiments;

FIG. 2 is an illustration of an unexpanded, cut embolic filter frame, according to some embodiments;

FIG. 3 is an illustration of a frame and filter of an embolic filter system, according to some embodiments;

FIG. 4 is an illustration of an embolic filter frame while flexing and an expanded view of an illustration of a flexion element on an embolic filter frame, according to some embodiments;

FIG. 5 is a flow chart of a method of assembling an embolic filter system, according to some embodiments;

FIGS. 6A to 6D are illustrations of a method of assembling an embolic filter system, according to some embodiments;

FIG. 6E to 6F are illustrations of a method of deploying an embolic filter system, according to some embodiments;

FIG. 7 is a flow chart of a method of assembling an embolic filter system, according to some embodiments;

FIGS. 8A to 8L are illustrations of a method of assembling an embolic filter system, according to some embodiments;

FIG. 9 is a flow chart of a method of implanting an embolic filter system, according to some embodiments;

FIGS. 10A to 10C are illustrations of flexion elements, according to some embodiments;

FIG. 11 is an illustration of an embolic filter frame with flexion elements along struts of the embolic filter; and

FIG. 12 is an illustration of the embolic filter of FIG. 12, wherein the embolic filter includes a sealing portion fully deployed and a filter portion that is still partially constrained.

### DETAILED DESCRIPTION

This disclosure is not meant to be read in a restrictive manner. For example, the terminology used in the application should be read broadly in the context of the meaning those in the field would attribute such terminology.

Persons skilled in the art will readily appreciate that various aspects of the present disclosure can be realized by any number of methods and apparatus configured to perform the intended functions. Stated differently, other methods and apparatus can be incorporated herein to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Certain relative terminology is used to indicate the relative position of components and features. For example, words such as “top”, “bottom”, “upper,” “lower,” “left,” “right,” “horizontal,” “vertical,” “upward,” and “downward” are used in a relational sense (e.g., how components or features are positioned relative to one another) and not in an absolute sense unless context dictates otherwise. Similarly, throughout this disclosure, where a process or method is shown or described, the method may be performed in any order or simultaneously, unless it is clear from the context that the method depends on certain actions being performed first.

With respect to terminology of inexactitude, the terms “about” and “approximately” may be used, in certain instances, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example.

As used herein, “couple” means join, connect, attach, adhere, affix, or bond, whether directly or indirectly, and whether permanently or temporarily.

As used herein, the term “elastomer” refers to a polymer or a mixture of polymers that has the ability to be stretched to at least 1.3 times its original length and to retract rapidly to approximately its original length when released. The term “elastomeric material” refers to a polymer or a mixture of polymers that displays stretch and recovery properties similar to an elastomer, although not necessarily to the same degree of stretch and/or recovery. The term “non-elastomeric material” refers to a polymer or a mixture of polymers that displays stretch and recovery properties not similar to either an elastomer or elastomeric material, that is, considered not an elastomer or elastomeric material as is generally known.

Various aspects of the present disclosure are directed toward an embolic filter device, system, and method. An exemplary embolic filter system 1000 is illustrated in FIG. 1. The embolic filter system 1000 generally includes a filter 1100 and an elongate element 1200. In various examples, the embolic filter system 1000 is configured such that the filter 1100 and the elongate element 1200 can freely articulate relative to one another (e.g., up to a 45 degree bend between the elongate element 1200 and the filter 1000). As discussed in greater detail below, in some examples, the filter 1100 includes one or more features that facilitate relative articulation along a longitudinal length of the filter 1100. Articulation along the filter 1100 facilitates deployment of the filter 1100 in tortious or otherwise difficult anatomy (e.g., lumens such as an artery or vessel). In some examples, the filter 1100 includes one or more features that facilitate articulation along the length of the filter 1100, while in other examples, the embolic filter system 1000 includes one or more additional components, such as one or more elements that facilitate such relative articulation along the length of the filter 1100. Providing an embolic filter system 1000 having a filter 1100 that is operable to articulate along the length of the filter 1100 provides that the embolic filter system 1000 can passively orient itself to achieve proper alignment of the filter 1100 relative to the vasculature within which it is partially or fully deployed. Alternatively, the embolic filter system 1000 can also be manipulated in situ by the operator to achieve such alignment.

As shown in FIG. 1, the embolic filter system 1000 includes a first end 1002 and a second end 1004. In some examples, the filter 1100 extends distally from the elongate element 1200 such that a first end 1102 of the filter 1100 defines, at least in part, the first end 1002 of the embolic filter system 1000. Similarly, in some examples, the elongate element 1200 includes a first end 1202 and a second end 1204, the elongate element 1200 extending from the filter 1100 such that the second end 1204 of the elongate element 1200 defines, at least in part, the second end 1004 of the embolic filter system 1000. In various examples, a second end 1104 of the filter 1100 is coupled with the elongate element 1200. In some examples, the second end 1104 of the filter 1100 is coupled with a first end 1202 of the elongate element 1200. In some examples, coupling the second end 1104 of the filter 1100 with the first end 1202 of the elongate element 1200 includes coupling the second end 1104 of the filter 1100 with the first end 1202 of the elongate element 1200 such that the first end 1202 of the elongate element 1200 is situated distal to the second end 1104 of the filter 1100 (e.g., such that the filter 1100 and the elongate element 1200 partially overlap one another).

In various examples, the embolic filter system 1000 can be used in combination with one or more auxiliary systems. For example, as shown in FIG. 1, one or more auxiliary systems 2000 including one or more auxiliary components may be utilized in combination with the embolic filter system 1000. In some examples, the auxiliary system 2000 and/or components thereof may be commercial-over-the-shelf (COTS) systems or components. One non-limiting auxiliary system 2000 includes a COTS catheter. Other non-limiting auxiliary systems 2000 include constraining sheaths, including tear-away sheaths, valves and connectors such as those used in controlling fluid backflow through one or more of the embolic filter system 1000 and the auxiliary system 2000 (e.g., Tuohy-Borst Connector(s)), and control handles. The auxiliary system 2000 may be used in association with one or more of the delivery, deployment, operation, and/or removal of the embolic filter system 1000. It is to be appreciated that, in various examples, the embolic filter system 1000 may, itself, include one or more of such tear-away sheaths, connectors, and/or valves, such as hemostatic valves.

The embolic filter system 1000 is generally configured to be advanced to a target site within a patient's vasculature such that one or more components of the embolic filter system 1000 (such as the filter 1100) is antegrade or “downstream” of a treatment area of the vasculature, between the treatment area and one or more anatomical regions where the presence of embolic debris can lead to complications and damage to the anatomy. Those of skill will appreciate that positioning the system downstream from the treatment area provides that embolic and other debris dislodged from the treatment area during a treatment procedure will migrate with the flow of blood toward the embolic filter system 1000 where the embolic debris can be filtered from the blood.

Properly orienting the filter 1100 of the embolic filter system 1000 within a vessel or region of the vasculature is an important factor in facilitating a proper deployment and successful filtering of embolic debris from the blood in association with an endovascular procedure. However, in certain portions of the vasculature and/or under certain conditions, it is difficult to deploy embolic filters such that they are operable to successfully filter embolic debris from the blood. The embolic filter system 1000 disclosed herein passively aligns itself along a surface of the vasculature such as a vessel wall to cause a relative articulation along a length of the filter 1100, thus achieving a proper alignment of the filter 1100 within the vasculature. Alignment within the vasculature generally results in a minimization of gaps between the filter 1100 and the vessel wall that could operate as avenues through which the embolic debris can bypass the embolic filter system 1000. Though, in some embodiments, the embolic filter system 1000 also affords the operator the ability to deploy the embolic filter system 1000 and then manipulate the embolic filter system 1000 to properly align the filter 1100 with the vasculature.

In various examples, articulation is achieved by one or more of advancement and withdrawal of the elongate element 1200 with the filter 1100 partially deployed. For example, advancement and/or withdrawal of the elongate element 1200 while the filter 1100 is partially deployed within the vasculature may operate to impart a compressive or tensile load to one or more of the filter 1100 and the elongate element 1200. As mentioned above, in various examples, the filter 1100 may include one or more features that facilitate relative articulation along the length of the filter 1100, while in other examples, the embolic filter system 1000 includes one or more additional components, such as one or more elements that facilitate relative articulation along the length of the filter 1100. In various examples, applying compressive and/or tensile load(s), the embolic filter system 1000 causes the one or more features of the filter 1100 and/or the one or more additional components to bend, deflect, or otherwise cause deformation thereof to achieve the relative articulation along the length of the filter 1100.

Once deployed, the embolic filter system 1000 interacts with blood flowing through the region of the vasculature within which the embolic filter system 1000 is deployed. In some examples, the embolic filter system 1000 may be adapted or otherwise configured to filter blood and/or embolic debris as it flows through or otherwise interacts with the embolic filter system 1000. In some examples, the embolic filter system 1000 additionally or alternatively redirects blood flow and/or embolic debris from what would otherwise be a normal or unimpeded flow of blood and/or embolic debris through the surrounding vasculature. Thus, in various examples, the embolic filter system 1000 can be deployed within a region of a patient's vasculature such that blood and/or embolic debris is filtered and/or redirected as it flows through that region of the patient's vasculature.

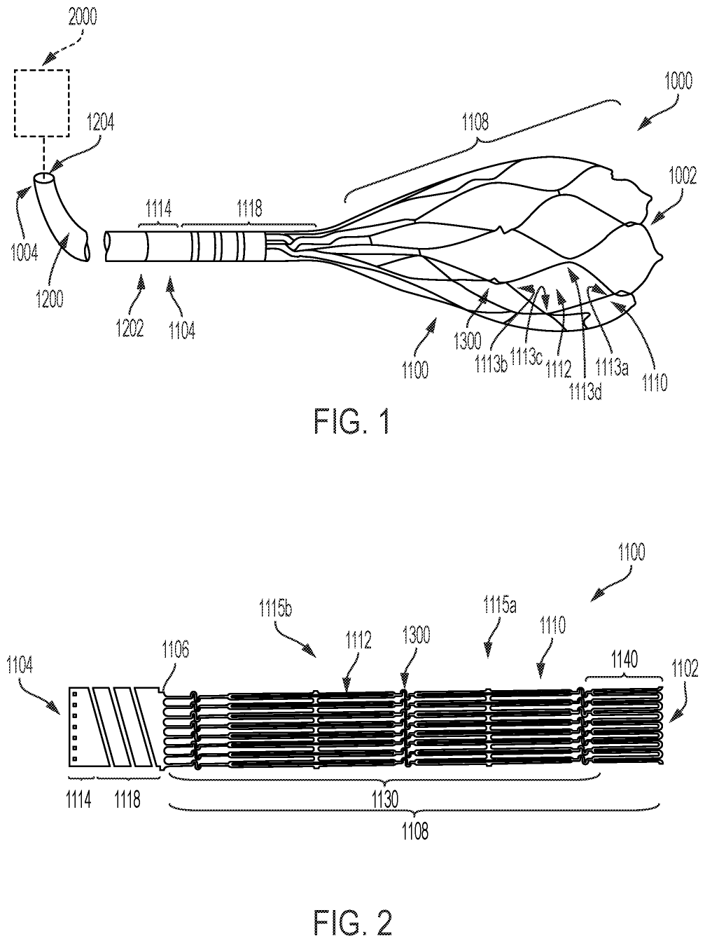

With reference now to FIGS. 1 and 2, the filter 1100 of the embolic filter system 1000 includes a body 1106 having the first and second ends 1002 and 1004. The filter 1100 generally includes a structural element forming a capture section 1108, an attachment section 1114, and an articulation section 1118. In some examples, the articulation section 1118 is intermediate to the first and second ends 1102 and 1104, and thus may be referred to as an intermediate section. FIG. 2 is a 2-dimensional plan view of the filter 1100 showing the full circumference of the filter 1100, which has been unwrapped and laid flat to illustrate the relationship between the capture section 1108, the attachment section 1114, and the articulation section 1118.

In various examples, the filter 1100 is a structure configured to interact with blood and/or embolic debris flowing through the patient's vasculature in the region within which the embolic filter system 1000 is deployed. As discussed in greater detail below, the filter 1100 or one or more portions thereof may be formed from a cut tube, a wire frame, a molded or extruded part, or a combination thereof. In some examples, one or more portions of the filter 1100 may be formed of a shape-memory material such as nitinol, such that the one or more portions thereof possess or exhibit self-expanding properties as would be appreciated by those of skill in the art. In other examples, however, one or more of the components of the filter 1100 may be formed from other resilient metals that may be expandable through the use of an expansion aid (such as a balloon). For example, one or more of the support elements may be formed from a polymer or a biocompatible metallic alloy such as stainless steel. In some examples, the filter 1100 or one or more portions thereof may be constructed of a durable elastomeric material, such as polyurethane or densified nylon.

As shown in FIGS. 1 and 2, the filter 1100 includes a capture section 1108 (also referred to herein as a structural element). The capture section 1108 is configured to direct or funnel blood and embolic debris into an interior region of the filter 1100 and, in some examples, the elongate element 1200. The capture section 1108, therefore, operates as an obstruction to the flow of blood that causes the blood to interact with the embolic filter system 1000 before flowing downstream of the embolic filter system 1000. In various examples, the capture section 1108 is configured to transition between a contracted configuration (e.g., FIG. 2) and an expanded configuration (e.g., FIG. 1) in conjunction with the embolic filter system 1000 transitioning from a delivery configuration to a deployed configuration such that the embolic filter system 1000 can be delivered endovascularly (e.g., at a small delivery profile), while possessing the capability of being deployed in situ to a larger deployed profile conducive for interrupting blood flow to filter embolic debris therefrom.

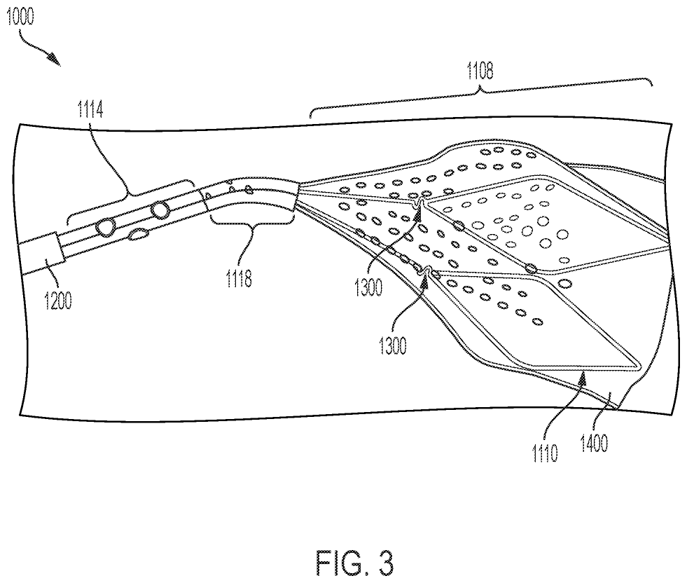

In the deployed configuration, the filter 1100 adopts a generally trumpeted, conical, or frustoconical shape in that a transverse cross-sectional area of the filter 1100 is different at two different longitudinal locations along the filter 1100 between the first and second ends 1102 and 1104 of the filter 1100. In some examples, the transverse cross-sectional area of the first end 1102 is greater than the transverse cross-sectional area of the second end 1104. In some examples, the filter 1100 generally tapers from the first end 1102 to the second end 1104 as shown in FIGS. 1 and 3, for example. Such a configuration provides that the filter 1100 operates to funnel the blood into the filter 1100 and/or into the elongate element 1200 as disclosed herein.

In various examples, the capture section 1108 is comprised of one or more support elements, such as one or more braids, meshes, lattices, wires, rings, struts, or any other suitable support element. For example, as shown in FIGS. 1 and 2, the capture section 1108 includes a plurality of strut elements 1110 that are arranged to define one or more closed cells 1112 that collectively define, at least in part, the capture section 1108. As shown, these closed cells 1112 are arranged in one or more rows (e.g., 1, 2, 3, 4, or more than 4 rows). It is to be appreciated, however, that braids, meshes, lattices, wires, rings, and other suitable support elements may be utilized in lieu of or in combination with the strut elements 1110, provided that the capture section 1108 of the filter 1100 is operable to transition between the contracted and expanded configurations.

In some examples, the closed cells 1112 are configured to change shape to accommodate or facilitate the transition of the structural element of the capture section 1108 between the expanded and contracted configurations. When the structural element of the capture section 1108 is in the expanded configuration, for example, the closed cells may be quadrilateral-shaped (e.g., diamond-shaped) as shown in FIG. 1. It will be appreciated, however, that the shape of the closed cells shown herein is not to be construed as limiting, and that various alternative shapes (e.g., polygonal) and/or sizes are envisioned.

It is also to be appreciated that the number of rows of closed cells and/or the number of closed cells per row may be increased or decreased to achieve a desired expanded profile (e.g., deployed diameter) and a desired contracted profile (e.g., delivery diameter), and thus the examples illustrated herein are not to be construed as limiting. Generally, for a given closed cell size and shape, increasing the number of closed cells 1112 increases the expanded and contracted profile diameters, and decreasing the number of closed cells 1112 decreases the expanded and contracted profile diameters. Similarly, for a given closed cell size and shape and number of closed cells 1112 per row, increasing the number of rows of closed cells 1112 increases a length of the filter 1100, and decreasing the number of rows of closed cells 1112 decreases the length of the filter 1100.

In some embodiments, the closed cells 1112 include a substantially elongate shape when the filter 1100 is in an expanded configuration (e.g., substantially in the shape of a diamond). Each closed cell 1112 includes apices 1113 defining the furthest most extent of the closed cell 1112 in a direction. For example, when the closed cells 1112 are substantially in the shape of a diamond, each closed cell includes a first longitudinal apex 1113a, a second longitudinal apex 1113b, a first lateral apex 1113c, and a second lateral apex 1113d. The first and second lateral apices 1113c, 1113d represent the interfacing of closed cells 1112 within the same row, and the first and second longitudinal apices 1113a, 1113b represent where the closed cells 1112 interfacing of a corresponding closed cell in an adjacent row, when an adjacent row exists. By way of example, filter 1100 may include a plurality of rows of closed cells 1112 such as a first row 1115a and a second row 1115b. The first row 1115a includes a first closed cell 1112a and the second row 1115b includes a second closed cell 1112b. Each of the closed cells 1112a, 1112b has a first longitudinal apex 1113a and a second longitudinal apex 1113b, wherein the first longitudinal apex 1113a is closer to the first end 1102 relative to the second longitudinal apex 1113b and second end 1104, and the second longitudinal apex 1113b is closed to the second end 1104 relative to the first longitudinal apex 1113a and the first end 1102. The first closed cell 1112a is adjacent to the second closed cell 1112b along the longitudinal length of the filter 1100. The second longitudinal apex 1113b of the first closed cell 1112a is positioned proximate the first longitudinal apex 1113a of the second closed cell 1112b.

In various examples, the capture section 1108 includes one or more features that are configured to facilitate flexion of the filter 1100 along its longitudinal length. Those features define a flexion zone 1130 along at least a portion of the length of the filter 1100. For example, as shown in FIGS. 1-4, the filter 1100 includes flexion elements 1300 that are configured to allow the filter 100 to bend or flex along the flexion zone 1130 with respect to a longitudinal axis. The flexion elements 1300 allows the filter to navigate and be deployed in tortious anatomy. In some embodiments, a sealing zone 1140 is also defined on the filter 1100, the sealing zone 1140 operable to interface with a patient's anatomy and form a removable seal between the filter 1100 and the patient's anatomy. The flexion zone 1130 and the sealing zone 1140 facilitate optimal interfacing between the filter 1100 and a tortious anatomical structure. For example, the filter 1100 can be deployed in a lumen (e.g., subclavian artery near the aortic arch) such that the filter flexes or bends at the flexion zone 1130 such that the filter 1100 is operable to form an effective seal with the surrounding anatomy and conform to the profile of the surrounding anatomy without straightening or straining the natural curvature or profile of the surrounding anatomy.

In some embodiments, the flexion zone 1130 includes at least one flexion element 1300 that facilitates bending or flexing of the filter 1100. For example, the flexion elements 1300 can space or partially mechanically isolate portions of the filter 1100 such that portions of the filter 1100 are partially buffered from each other to reduce levels of force transmitted to adjacent portions of the filter 1100. In some embodiments, flexion elements 1300 extend between adjacent closed cells 1112. The flexion elements 1300 may extend from apices 1113 of the closed cells 1112. For example, the flexion element 1300 extends between a second longitudinal apex 1113b of a first closed cell 1112a and a first longitudinal apex 1113a of a second closed cell 1112b. By placing a flexion element 1300 between the first and second cells 1112a, 1112b are partially mechanically isolated or buffered from the movement of the other. Furthermore, the partial mechanical isolation of the closed cells 1112 by the flexion elements 1300 allows the filter 1100 to maintain a more consistent diameter during flexion. For example, because the bending or flexion is mainly occurring at the flexion elements 1300, the closed cells (e.g., diamond-shaped closed cells), are not compressed in order to achieve the bend and therefore do not radial expand in response to the compression. This allows the filter 1100 to maintain a more consistent desired diameter when deployed.

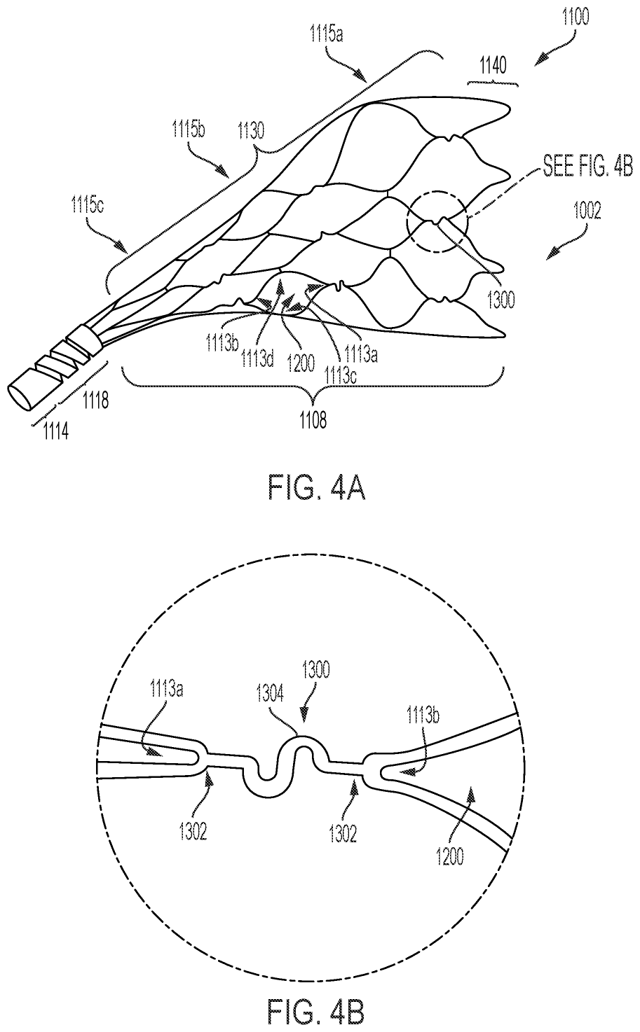

In some embodiments, the flexion element 1300 is operable to extend and contract, allowing the cell rows 1115 to be positioned more closely together or further apart. Furthermore, each of the flexion elements 1300 is independent from every other flexion element 1300. This facilitates the bending or curving of the filter 1100 along the longitudinal length of the filter 1100 without kinking the body of the filter 1100 or causing folds or creases to form along the longitudinal length of the filter 1100. For example, a filter 1100 may include a plurality of flexion elements 1300 extending between closed cells 1112 of a first row 1115a and closed cells 1112 of a second row 1115b (as well as a third row 1115c, and so forth). A portion of the flexion elements 1300 may be partially or fully extended about an arc length of the filter 1100 and another portion of the flexion elements 1300 may be partially or fully compressed about another arc length of the filter 1100, allowing the filter to bend or flex along a longitudinal length (e.g., as shown in FIG. 4). In some embodiments, the flexion elements 1300 being fully compressed and/or fully elongated serves to act as a limit or stop to further bending or curving of the filter 1100 absent a substantial increase in bending force. Thus, the flexion elements 1300 facilitate mechanical isolation of rows 1115 of cells 112 allowing the rows 1115 to “flex” independently (e.g., in an undulating vessel).

In further embodiments, the flexion element 1300 is operable to articulate in 360 degrees about position where the flexion element 1300 is coupled to the closed cells 1112. For example, in those embodiments in which the filter 1100 includes a first row 1115a and a second row 1115b of closed cells 1112, the flexion elements 1300 are able to articulate 360 degrees within a plane transecting the filter 1100 at a position along the longitudinal length of the filter 1100. This further allows the filter 1100 to conform to the anatomy of the patient, especially tortious anatomy, by providing the rows 1115 the ability to pivot, bend, or otherwise flex responsive to the surface and three dimensional profiled of the patient's anatomy. Because the flexion elements 1300 can articulate as described, bends or flexion may be achieved along the longitudinal length of the filter 1100 that are relatively abrupt in order to accommodate specific anatomies, for example, the aortic arch.

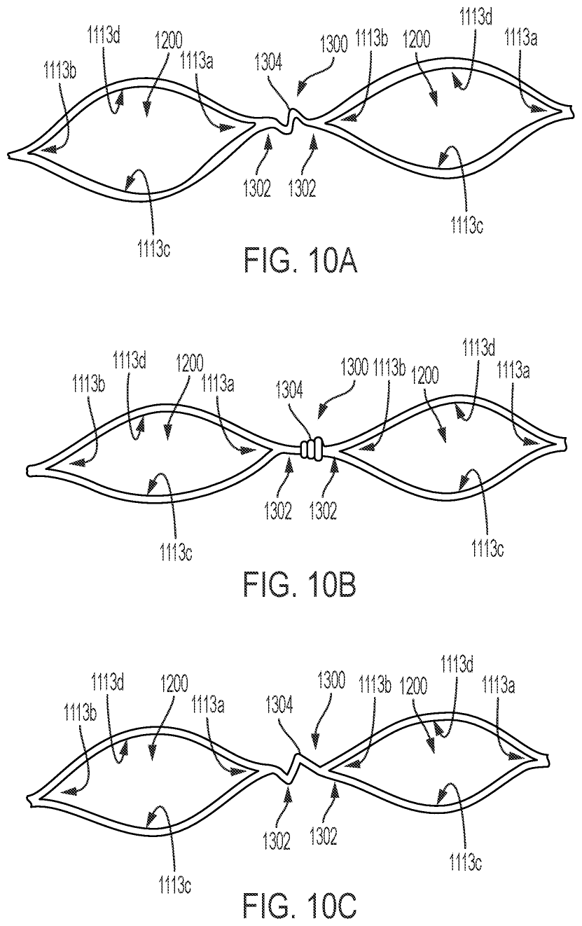

In various examples, the flexion element 1300 includes features for facilitating flexion of the filter 1100 and partial mechanical isolation of the closed cells 1112. The features for facilitating flexion may allow the flexion element 1300 to bend, flex, extend, compress, and otherwise articulate. For example, the flexion element 1300 includes a serpentine-shaped extension which can be elastically deformed to various shapes, lengths, and angles. The serpentine-shaped extension can include the wavy or serpentine path shown (e.g., approximately sinusoidal) in FIG. 4. For example, the flexion element 1300 may include a coupling portion 1302 and a flexion portion 1304. The coupling portion 1302 of the flexion element 1300 interfaces with the strut elements 1110 defining the cells 1112 and the flexion portion 1304 includes the serpentine-shaped extension. The serpentine-shaped extension includes a first portion that extends generally in a first direction and a second portion that bends about 180 degrees and extends in a second, opposite direction. In other embodiments, the flexion portion 1304 can include a zig-zap shape, a coil shape, or another shape operable to allow the flexion element 1300 to articulate in various directions. In other embodiments, the flexions elements 1300 may include various other shapes, for example, zig zag (e.g., as seen in FIG. 10C), coil or helical (e.g., as seen in FIG. 10B), curved, U-shaped, C-shaped, and any other shape that facilitates adjustment of the position of two apices 1113a, 1113b of adjacent closed cells 1112a, 1112b.

In various examples, the particular aspects or features of the flexion element 1300 (e.g., the tightness of the turn, the length of the segments or portions, the size of the slots and distance therebetween) is selected to provide that the flexion element 1300 and facilitate articulation of the cells 1113 relative to one another by a designated amount. For instance, the particular aspects or features of the flexion element 1300 (such as the radius of the turn of the serpentine-shaped or helical-shaped flexion elements 1300) can be configured such that the capture section 1108 can be articulated such that a relative angle defined between the longitudinal axes thereof (i.e., an articulation angle) is up to 30 degrees, up to 45 degrees, up to 60 degrees, up to 90 degrees, or up to 180 degrees. These relative angles are not intended to be limiting but are instead intended to be exemplary. For instance, the flexion element 1300 can be configured to adopt an articulation angle of up to between 90 and 180 degrees. Additionally or alternatively, in some examples, a length of the flexion element 1300 can be varied to increase, decrease, or otherwise alter the number, shape, and configuration of the particular aspects or features facilitating articulation (e.g., no. of turns, pitch of portions, slot width), and thereby alter the degree of passive articulation. In is appreciated that the flexion elements 1300 are capable of providing greater flexion between the cells 1112, however, may exhibit less flexion in practice than is described herein because of each of the cells 1112 being coupled at various other positions (e.g., lateral apices 1113c, 1113d) which constrain the travel of the cells 1112 relative to each other.

In various examples, the flexion elements 1300 may be positioned about the capture section 1108 in various patterns. For example, in some embodiments, the flexion elements 1300 may be positioned between each closed cell 1112. In other embodiments, the flexion elements 1300 may be positioned periodically between closed cells 1112, which can provide increased flexion relative to a filter not implementing a flexion element, but providing less flexion relative to a filter having flexion elements 1300 between each cell 1112. In some embodiments, the flexion elements 1300 are positioned on one section of a filter 1100. For example, when a filter 1100 is to be used in connection with the aortic arch, the filter 1100 may include flexion elements 1300 positioned on one surface of the filter 1100 so as to promote flexion of the capture section 1108 in a first direction.

It is to be appreciated that the flexion elements 1300 are operable to allow two apices 1113a, 1113b of adjacent closed cells 1112a that are operably coupled to each other by the flexion element 1300 to articulate with respect to each other. In some embodiments, the flexion element 1300 is operable to allow the apices 1113a, 1113b to move with respect to each other in three dimensions (e.g., movement with respect to each other are not constrained to specific planes). For example, the apices 1113a, 1113b are operably coupled to each other via the flexion element 1300 such that the apices are operable to move away from and toward each other in the longitudinal directions (e.g., the flexion element 1300 is operable to expand and compress/contract along or parallel to the longitudinal axis. Furthermore, the flexion elements 1300 are operable to bend and articulate such that the closed cells 1112 are out of plane with each other (e.g., planes defined by the closed cells are angled relative to each other at an angle greater than zero). Likewise, the flexion elements 1300 are operable to bend and articulate such that the closed cells remain in the same plane, but are operable to bend such that a longitudinal axis defined between two longitudinal apices 1113a, 1113b of a first closed cell 1112a and a longitudinal axis defined between two longitudinal apices 1113a, 1113b of a second closed cell 1112b are angled relative to each other (e.g., greater than zero degrees). Stated otherwise, the flexion elements 1300 are operable to extend, collapse, and bend, where the bending is can occur in any direction, that is, in 360 degrees about each of the connection positions with the apices 1113 of the closed cells 1112.

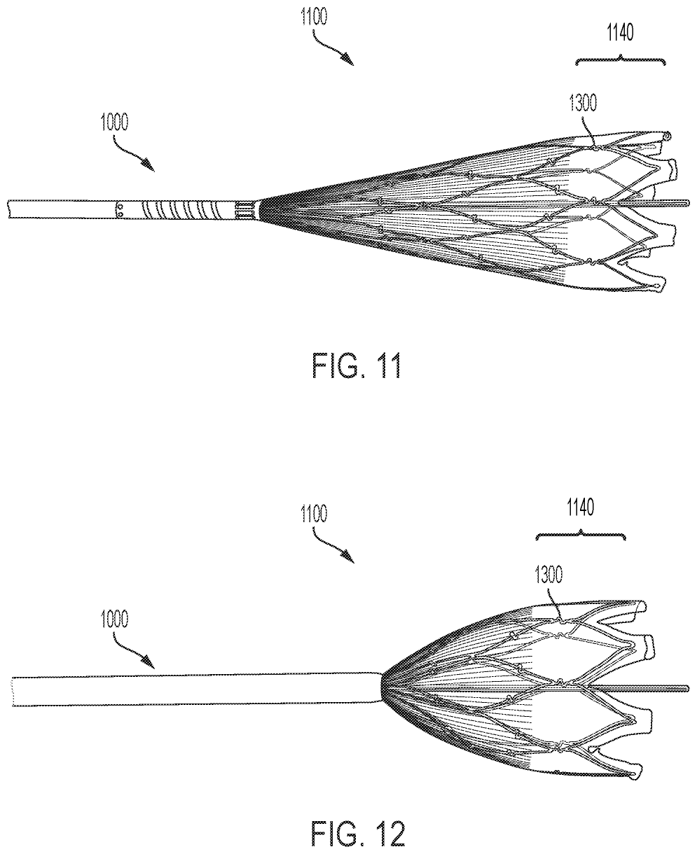

Referring to FIG. 11, the flexion element 1300 may be incorporated at other positions in the flexion zone 1130, for example along the strut elements 1110 that are arranged to define the closed cells 1112. In one embodiment, the flexion elements 1300 may be incorporated into the strut elements 1110 along the length of the strut element. The struts element 1130 may include a first end 1131a and a second end 1131b, the flexion element 1300 being positioned between the first end 1131a and the second end 1131b. Thus, the flexion element 1300 may be positioned along an edge of the closed cells 1112 instead at an end or apex of the closed cells 1112. This provides increased flexion or conformability of the filter 1100 to by including more positions at which the filter 1100 is able to pivot or flex along the longitudinal length of the filter 1100. Increased numbers positions of the flexion elements 1300 along the length of the filter 1100 also provides increased stored length for navigating tight turns at the target site. It is understood that any number of flexion elements 1300 may be implemented along the length of the filter 1100 and in various combinations and configurations. For example, flexion elements 1300 may be positioned at the apex of each closed cell 1112 in a row 1115 and along the length of each strut element 1110 of each of the closed cells 1112, flexion elements 1300 may be positioned at some apices of the closed cells 1112 and along the length of some of the strut elements 1110 of the closed cells, or a combination thereof. The flexion of the filter 1100 is increased by having an increased number of flexion elements 1300 positioned on the filter 1100.

When flexion elements 1300 are incorporated along the length of the strut elements 1110, the flexion elements 1300 may be generally circumferentially aligned with each other at a longitudinal position along the filter 1100, thus forming a flexion element row 1306. The flexion elements 1300 within a flexion row 1306 may be slightly staggered in order to facilitate compaction of the filter 1100 into a delivery configuration. However, even when the flexion elements 1300 are slightly staggered, the flexion elements 1300 are still positioned within a circumferential zone that facilitates flexion of the filter 1100. Referring to FIG. 12, by incorporating flexion elements 1300 along the length of the strut elements 1110 in addition to the incorporating flexion elements 1300 at the apices 1113 of the cells 1112, the filter 1100 is operable to be deployed at a shorter length. By having the additional flexion elements 1300, the filter 1100 is provided increased flexion allowing a transition of a longitudinal length of the filter 1100 to be shorter for a filter 1100 that is partially deployed such that one longitudinal end is still constrained and another longitudinal end is deployed. Because the transition region is shortened, the filter 1100 is operable to be deployed within a vessel such that a seal is created with the surrounding tissue (e.g., a vessel wall) while the a portion of the filter 1100 is still in the constrained configuration. This allows the filter 1100 to be deployed and operational in positions in which the vessel profile limits or restricts deployment of the filter 1100 along the entire longitudinal length of the filter 1100. Furthermore, the operational length of the filter 1300 is increased in that the range of deployment is increased. More specifically, in embodiments not implementing flexion elements, the filter 1100 may need to be unconstrained over at least 2-3 cm in order for the filter 1100 to expand to a sufficient diameter to contact the surrounding tissue and form a seal with that tissue. However, embodiments implementing the flexion elements 1300 as described significantly decrease the length of filter 1100 that is unconstrained in order to seal with the tissue as compared to those without flexion elements 1300. For example, those embodiments using flexion elements 1300 are operable to facilitate deployment of the filter 1100 such that the filter 1100 is operable to expand to its expanded state for at least a portion of its longitudinal length when a first length of the filter 1100 is unconstrained where the first length is equal to about the length of about 1.5 cells 1112. It is understood that the ratio may vary some with the diameter of the filter 1100 as it increases or decreases relative to those illustrated herein.

In some embodiments, the flexion element 1300 is formed of a shape-memory material. The shape-memory properties of the flexion element 1300 facilitates the flexion element 1300 returning to a predetermined shape or configuration. The flexion element 1300 is operable to elastically deform and return to a neutral configuration. For example, the flexion elements 1300 are configured to elastically deform under normal operating conditions (e.g., where the flexion elements 1300 is configured to elastically deform to accommodate a maximum expected articulation during a given endovascular procedure). By configuring the flexion elements 1300 to elastically deform under expected operating conditions (e.g., an expected degree of angulation), the embolic filter system provides that the filter 1100 can be articulated in a resilient manner such that the flexion elements 1300 resiliently returns to its pre-set shape upon removal of the force required to cause the articulation. Such a configuration provides that the embolic filter system 1000 is in linear alignment for collapse and removal following an endovascular procedure. In some embodiments, the flexion elements 1300 are non-rigid so as to allow relative articulation of the structural elements forming the capture section 1108 from which the flexion elements 1300 extend.

In those embodiments in which the flexion element 1300 is formed of a shape-memory material, the force required to deform the flexion element 1300 may be less than the force required to deform the structural element forming the capture section 1108, allowing the structural element to return to a shape-set configuration against forces that may continue to deform the flexion element 1300. For example, when a filter 1100 is being deployed in situ, the structural elements forming the capture section 1108 may be deployed from a constrained configuration within a delivery device toward an expanded configuration. The flexion elements 1300 may be elastically deformed or deflected as portions of the structural elements forming the capture section 1108 are self-expanding while other portions are maintained in the constrained configuration. Because the flexion elements 1300 are operable to extend, contract, and articulate, the capture section 1108 is operable to deploy further towards the delivery configuration with decreased resistance to deployment as compared to embodiments in which the structural elements of the capture section 1108 forming a first closed cell 1112a are coupled directly to structural elements forming a second closed cell 1112b. Stated otherwise, a row 1115 of closed cells 1112 is operable to deploy to the deployed configuration or to secure contact with the target tissue more quickly (e.g., with less deployment of the filter 1100 as whole) relative to when the rows 1115 of closed cells 1112 are coupled directly together at their apices 1113. This can be described as “flowering” as the filter 1100 is deployed toward the deployed configuration.

Because the flexion elements 1300 are operable to allow the filter 1100 to bend or flex along its longitudinal length, the filter 1100 is operable to be deployed in a target lumen orthogonally. For example, when the lumen includes a substantially circular cross section when taken perpendicular to a longitudinal axis, the filter 1100 is operable to be deployed such that a cross section of the filter 1100 when taken perpendicular to the filter's longitudinal axis is likewise circular. Stated otherwise, when deployed, the filter 1100 conforms to the shape of the lumen such that the longitudinal axis of each are similarly positioned (e.g., the lumen and the filter 1100 are coaxial). This is especially important at the first end 1102 of the filter 1100 such that a functional seal is formed and all of the fluid flowing through the lumen is flowing through the filter 1100. This is further demonstrated in importance in anatomy that is tortious or is an intersection of various lumens (e.g., near ostia such as present in the aortic arch.

In some embodiments, the structural elements of the capture section 1108 from which the flexion elements 1300 extend may not form a fully closed cell. For example, the structural elements may form a partial cell when positioned at the first end 1102 of the filter 1100. Flexion elements 1300 are not limited to being positioned between closed cells 1112 but may extend between partial cells or between a closed cell 1112 and a partial cell. Furthermore, flexion elements 1300 are not limited to extending from longitudinal apices 1113a, 1113b of cells, but may also extend from lateral apices 1113c, 1113d formed by the structural elements of the capture section 1108. Thus, the flexion elements 1300 may couple cells within a row 1115 as well as between different rows 1115.

As mentioned above, the filter 1100 may include one or more shape memory alloys, and thus may include one or more sections that are expandable. Thus, in various embodiments, the filter 1100 is configured to transition between a delivery configuration and a deployed configuration, where one portion of the filter 1100 is expanded relative to another portion of the filter 1100. For instance, in the delivery configuration, each of the various sections of the filter 1100 exhibit a profile (e.g., a diameter) adapted for delivery through a patient's vasculature, such as through or within a delivery catheter as described further below. Conversely, in the deployed configuration, one or more of the various sections of the filter 1100 are expanded relative to one or more of the other various sections of the filter 1100. As shown in FIG. 1, the embolic filter system 1000 is shown in a deployed configuration, where the structural element of the capture section 1108 is expanded relative to each of the articulation section 1118, the attachment section 1114, and the elongate element 1200. In some examples, the filter 1100 is configured such that the structural element of the capture section 1108 is self-expandable. In other examples, however, the filter 1100 is configured such that the structural element of the capture section 1108 is expandable through the use of an expansion aid (such as a balloon).

In various examples, the elongate element 1200 is a longitudinally extending structure having a first end 1202 and a second end 1204. In some examples, the elongate element 1200 is configured to receive blood and/or embolic debris that is directed into the embolic filter system 1000 by the filter 1100. Accordingly, in some examples, the elongate element 1200 includes a lumen. In various examples, the elongate element 1200 is configured to be advanceable through the vasculature. Thus, the elongate element 1200 is generally flexible yet longitudinally stable and compressible without risk of kinking or buckling under loading conditions consistent with advancement through vasculature, including advancement through one or more delivery catheters. In some examples, the elongate element 1200 may include a braided, wrapped, or cut reinforcement member attached to a body portion of the elongate element 1200 as a framework to add stability to the structure of the elongate element 1200. A reinforcement member may be braided by weaving a plurality of wire strands made of a suitable material. Regardless, the reinforcement member (e.g., the wire(s) or filament(s) forming the reinforcement member) may be made of metal and metal alloys (e.g., nitinol), polymeric materials, elastomeric materials, natural materials, or combinations of any of the foregoing. The reinforcement member may be symmetrically braided (e.g. with an opposing bias in an over/under configuration to form a typical braid) or having an asymmetric bias, with each strand of the braided wire oriented at a pitch angle ranging from 0° to 10°, 10° to 20°, 20° to 30°, 30° to 40°, 40° to 50°, 50° to 60°, 60° to 70°, 70° to 80°, 80° to 90°, or any combination thereof, relative to a longitudinal axis of the braided wire.

The elongate element 1200 may therefore comprise various materials including but not limited to medical grade polymeric materials including thermoplastic polymers, organosilicon polymers, and polyamides. Polyether block amide (e.g., PEBAX®), Nylon, polytetrafluoroethylene (PTFE), and Stainless steel are suitable non-limiting examples. The elongate element 1200 may be formed according to known methods, such as extrusion. In some examples, the elongate element 1200 may include one or more reinforcement elements, such as one or more fibers or braids extending along or within the material of the elongate element 1200. For instance, in some examples, the elongate element 1200 may include coil reinforced Nylon or PEBAX.

In some examples, the elongate element 1200 may be formed using a high durometer material, in which the hardness of the elongate element 1200 may be from 50 to 60 Shore Hardness Units, 60 to 70 Shore Hardness Units, 70 to 80 Shore Hardness Units, 80 to 90 Shore Hardness Units, or any combination thereof. Such materials may include thermoplastics, for example but not limited to Polymethyl Methacrylate (PMMA or Acrylic), Polystyrene (PS), Acrylonitrile Butadiene Styrene (ABS), Polyvinyl Chloride (PVC), Modified Polyethylene Terephthalate Glycol (PETG), Cellulose Acetate Butyrate (CAB); Semi-Crystalline Commodity Plastics that include Polyethylene (PE), High Density Polyethylene (HDPE), Low Density Polyethylene (LDPE or LLDPE), Polypropylene (PP), Polymethylpentene (PMP); Polycarbonate (PC), Polyphenylene Oxide (PPO), Modified Polyphenylene Oxide (Mod PPO), Polyphenylene Ether (PPE), Modified Polyphenylene Ether (Mod PPE), Thermoplastic Polyurethane (TPU); Polyamides such as nylon-11 and nylon-12, Polyoxymethylene (POM or Acetal), Polyethylene Terephthalate (PET, Thermoplastic Polyester), Polybutylene Terephthalate (PBT, Thermoplastic Polyester), Polyimide (PI, Imidized Plastic), Polyamide Imide (PAI, Imidized Plastic), Polybenzimidazole (PBI, Imidized Plastic); Polysulfone (PSU), Polyetherimide (PEI), Polyether Sulfone (PES), Polyaryl Sulfone (PAS); Polyphenylene Sulfide (PPS), Polyetheretherketone (PEEK); Fluoropolymers that include Fluorinated Ethylene Propylene (FEP), Ethylene Chlorotrifluoroethylene (ECTFE), Ethylene, Ethylene Tetrafluoroethylene (ETFE), Polychlorotrifluoroethylene(PCTFE), Polyvinylidene Fluoride (PVDF), Perfluoroalkoxy (PFA), or combinations, copolymers, or derivatives thereof. Other commonly known medical grade materials include elastomeric organosilicon polymers and polyether block amide. In particular, polyamides can include nylon 12, nylon 11, nylon 9, nylon 6/9, and nylon 6/6. In certain embodiments, PET, nylon, and PE may be selected for medical balloons used in high pressure applications. In some embodiments, the elongate element 1200 may include a braid reinforced structure to improve burst pressure resistance. In some embodiments, the elongate element 1200 may include one or more layers of hydrophilic coatings or other types of low-friction coatings and/or liners to reduce friction forces on the surface thereof. The specific choice of materials depends on the desired characteristics or intended application of the balloon.

The aforementioned reinforcement member may be combined with the high durometer material to form the elongate element 1200 such that the body portion of the elongate element 1200 is reinforced while the end of the elongate element 1200 is inserted into the second end 1104 of the filter 1100. In some examples, the high durometer material helps facilitate bonding of the end of the elongate element 1200 to the second end 1104 (e.g., by facilitating greater flow and mechanical engagement during heating and/or by increasing frictional/stiction engagement). In some examples, the bonding may be assisted using an adhesive, such as the UV cured adhesive as previously explained.

In some examples, blood and/or embolic debris entering the elongate element 1200 flows through the lumen of the elongate element 1200, such as from the second end 1204 of the elongate element 1200 to the first end 1202 of the elongate element 1200. In some examples, one or more auxiliary systems 2000 may be fluidly coupled with the lumen of the elongate element 1200, such as at the first end 1202 of the elongate element 1200. In some such examples, such auxiliary systems 2000 may be operable to aspirate the contents of the lumen (e.g., embolic debris and/or blood) of the elongate element 1200.

In some examples, the lumen of the elongate element 1200 forms a working lumen through which one or more medical devices (e.g., guidewires, endoprostheses) can be passed to treatment areas proximate the embolic filter system 1000. Thus, in various examples, the lumen of the elongate element 1200 operates as both a working lumen for medical device delivery as well as a structure for redirecting the flow embolic debris and/or blood. In some examples, the working lumen of the elongate element 1200 may be in a range of 4 Fr to 26 Fr, or larger.

Examples of medical devices that may be passed through the lumen of the elongate element 1200 include but are not limited to catheters, thrombectomy devices, atherectomy devices, embolectomy devices, and tools associated therewith, contrasting agents, drug delivery agents, endovascular prostheses including stents, stent-grafts, and valves, for example.

In various embodiments, the embolic filter system 1000 includes a membrane disposed along one or more portions of the filter 1100, and optionally along one or more portions of the elongate element 1200. For example, as shown in FIG. 3, a membrane 1400 is disposed about an exterior of the capture section 1108 and the articulation section 1118 of the filter 1100. In these examples, by disposing the membrane 1400 along the capture section 1108, the membrane 1400 operates to filter and retain embolic debris within the embolic filter system 1000 that would otherwise be free to escape through the voids in the capture section 1108 (e.g., the closed cells 1112) and the articulation section 1118.

Under certain conditions, the forces required to withdraw the embolic filter system 1000 from the vasculature may be quite high (e.g., higher than the forces required to cause the articulation section 1118 to bend to facilitate articulation between the filter 1100 and the elongate element 1200). For instance, removal of the embolic filter system 1000 may include withdrawing the embolic filter system 1000 within a delivery catheter, which includes re-collapsing the deployed filter 1100 whereby the distal end of the delivery catheter operates as a bearing surface that causes the filter 1100 to radially collapse as the embolic filter system 1000 is withdrawn into a lumen of the delivery catheter.

It should be appreciated that the membrane 1400 may additionally or alternatively be disposed about an interior of the capture section 1108 and the articulation section 1118. In some examples, the membrane 1400 may optionally extend to cover a portion of the overlapping sections of the elongate element 1200 and attachment section 1114 of the filter 1100.

In some examples, the membrane 1400 operates to filter or otherwise condition the blood and embolic debris flowing into the embolic filter system 1000. In some examples, the membrane 1400 is permeable to certain blood media (e.g., blood-permeable) and impermeable to certain other blood media and/or embolic debris. Specifically, in some examples, the membrane 1400 is configured such that certain blood media (e.g., red blood cells, white blood cells, plasma, platelets, etc.) flowing into the embolic filter system 1000 can permeate the membrane 1400 of the filter 1100 and re-enter the vasculature while the membrane 1400 is impermeable to certain other blood media and embolic debris. In some examples, the membrane 1400 is impermeable to embolic debris of a designated size or larger. That is, in some examples, the membrane 1400 operates to obstruct embolic debris of a designated size or larger from permeating the membrane 1400 of the filter 1100 and re-entering the vasculature.

In some examples, the blood media and embolic debris flowing into the embolic filter system 1000 that does not permeate back into the vasculature is either captured and retained within the filter 1100 or is further directed into the elongate element 1200. In some examples, as explained in greater detail below, the filter 1100 is collapsible such that blood media and embolic debris captured within the filter 1100 can be subsequently removed with the removal of the embolic filter system 1000 from the vasculature.

In some examples, blood and/or embolic debris that is directed into the elongate element 1200 may be aspirated therefrom prior to removal of the embolic filter system 1000 from the vasculature. Evacuating embolic debris that is captured within the filter 1100 helps minimize the risk that the captured embolic debris will be unintentionally released back into the patient's vasculature during removal of the embolic filter system 1000 from the patient's vasculature. For example, a known risk during embolic debris filtering procedures is the risk of tearing the membrane 1400 of the filter 1100 during removal. Embolic filters that are filled with embolic debris generally occupy a larger cross-sectional area than do embolic filters free of embolic debris. This increased cross section can be associated with difficultly in sufficiently collapsing the embolic filter to a configuration wherein the embolic filter can be completely retracted within a delivery catheter. Even where the filter is not retracted within a delivery catheter, withdrawing a filter having a larger diameter as a result of being filled with embolic debris through tortuous vasculature can be difficult.

The membrane 1400 may comprise various materials including, but not limited to polymers such as fluoropolymers like an expanded polytetrafluoroethylene (“ePTFE”), expanded modified PTFE, expanded copolymers of PTFE, FEP, PFA, nylons, polyurethanes, polycarbonates, polyethylenes, polyester, silicone and silicone elastomers (e.g. SYLGARD™ 184), urethane, thermoplastic polyurethane, polypropylenes, and the like.

In various examples, one or more regions of such materials may be further or alternatively modified by forming one or more perforations therein to control the permeability of the material. For example, a material such as an expanded fluoropolymer (or another suitable polymer) can be further modified by perforating one or more regions of the material to achieve a designated porosity. Examples include laser cutting or laser drilling holes or perforations into a material. Other materials having a woven, knitted or lattice configuration may also serve as adequate materials based on their permeability/porosity. Moreover, a desired permeability may be achieved through increasing or decreasing layers of the membrane material, as those of skill will appreciate. Additionally or alternatively, the permeability of the membrane 1400 may be optimized by manipulating the microstructure of the membrane material. In some such instances, a node and fibril configuration of an expanded fluoropolymer can be modified/optimized to achieve desired permeability. For example, an expanded fluoropolymer can be processed such that a node and fibril configuration of the expanded fluoropolymer is generally impermeable to embolic debris (and other blood media) of a designated size consistent with the discussion below.

In some examples, the membrane material can be configured such that one or more portions or regions are permeable to a media up to a designated size while one or more other portions or regions are impermeable to the media of the designated size or larger. In some examples, the size of the pores or perforations (or voids in the node and fibril microstructure) present in the membrane material may vary, for example, from a proximal end to a distal end and/or at one or more discrete locations.

In various examples, the membrane 1400 may be configured such that the membrane 1400 is impermeable to embolic debris greater than or equal to about 140 μm. In some such examples, the average pore size (or perforation size or void size in the node and fibril microstructure of the membrane 1400) may be less than 140 μm. In other examples, the membrane 1400 may be configured such that the membrane is impermeable to embolic debris smaller than 140 μm, such as embolic debris in the range of 40 μm to 99 μm. Such examples are not intended to be limiting. For instance, if desired, the membrane 1400 may be configured to be permeable to embolic debris of 100 μm, 120 μm, 140 μm, 160 μm, 180 μm, 200 μm (or larger), and anywhere therebetween, in which case an average pore size (or perforation size or void size in the node and fibril microstructure of the membrane 1400) may exceed 150 μm.

In various embodiments, the embolic filter system 1000 is advanced to the treatment area within the vasculature in a delivery configuration, after which the embolic filter system 1000 is operable to be deployed or otherwise transitioned to a deployed configuration. In the delivery configuration, the embolic filter system 1000 is in a generally contracted configuration. In some examples, in the delivery configuration, the capture section 1108 is radially contracted such that the capture section 1108 is operable to be delivered endovascularly (e.g., at a small delivery profile), such as through a delivery catheter as discussed further below. In some examples, one or more of the articulation section 1118, the attachment section 1114, and one or more regions of the elongate element 1200 may additionally be radially contracted, though the same is not required. In the deployed configuration, the capture section 1108 is transitioned to a radially expanded configuration (e.g., FIG. 1) operable to interrupt blood flow to cause embolic debris to be filtered therefrom. In some examples, one or more of the articulation section 1118, the attachment section 1114, and one or more regions of the elongate element 1200 may additionally be radially expanded in the delivery configuration, though the same is not required.

After completion of the endovascular procedure, the embolic filter system 1000 is operable to be removed from the vasculature. In some examples, embolic debris captured by the embolic filter system 1000 may be aspirated or otherwise removed from the embolic filter system 1000 prior to removal of the embolic filter system 1000 from the vasculature, as mentioned herein. In some examples, to remove the embolic filter system 1000, the embolic filter system 1000 is transitioned from the deployed configuration to the delivery configuration. In some examples, such a transition from the deployed configuration to the delivery configuration includes a contraction of one or more portions of the embolic filter system 1000 (e.g., one or more portions of the filter 1100 and the elongate element 1200). For instance, in various examples, removal of the embolic filter system 1000 includes radially contracting or compressing the capture section 1108 of the filter 1100 to a profile (e.g., a diameter) conducive for endovascular removal. It is to be appreciated that a diameter of the structural element of the capture section 1108 is smaller when the embolic filter system 1000 is in the delivery configuration that when the embolic filter system 1000 is in the deployed configuration.

The embolic filter system 1000 is operable to be delivered to treatment areas within the vasculature in association with a variety of different delivery methods. As such, the embolic filter system 1000 is also operable to be assembled in a variety of different methods. The following discussion details various assembly and delivery methods associated with the embolic filter system 1000.

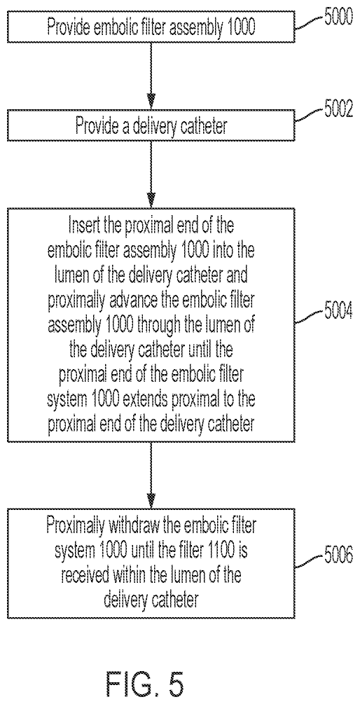

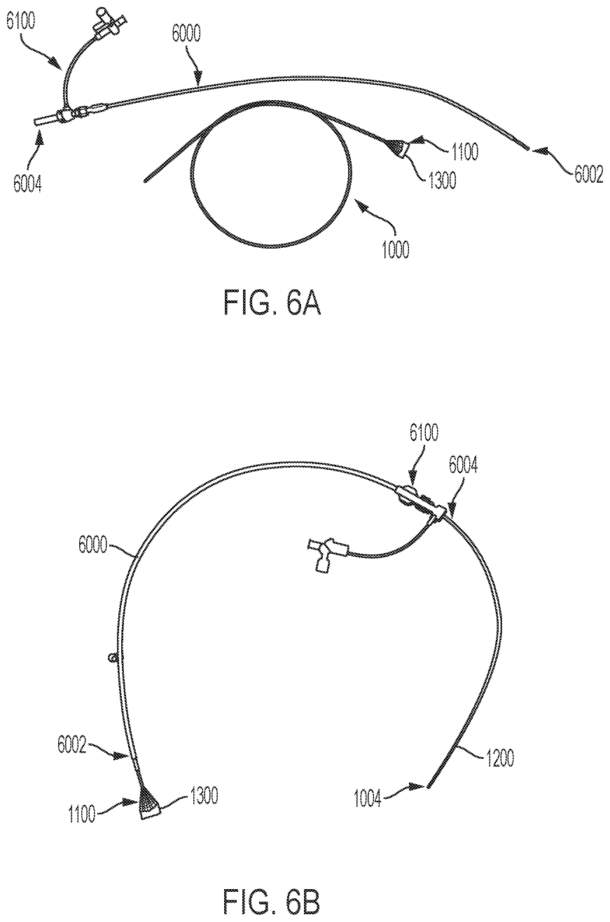

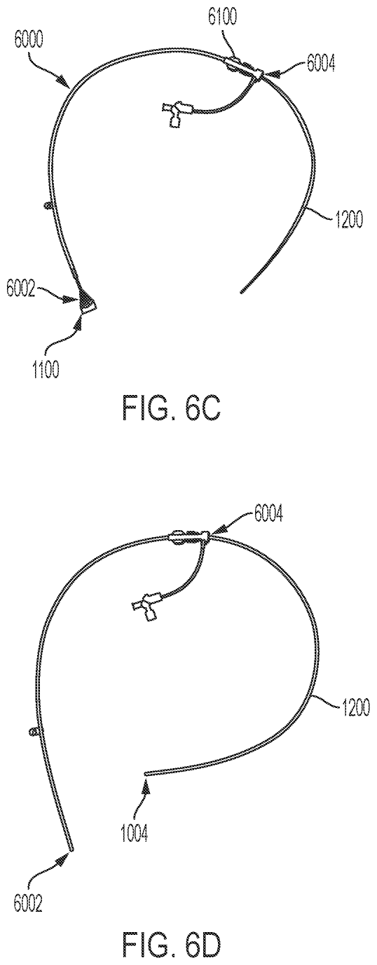

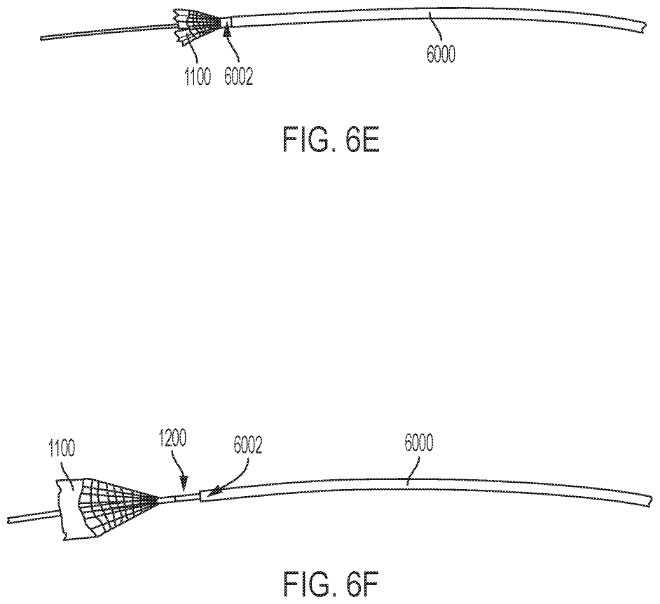

Turning now to FIG. 5, a flow chart is illustrated that outlines one example method for a medical device assembly including the embolic filter system 1000. As shown, step S000 includes providing the embolic filter system 1000. As discussed above, the embolic filter assembly generally includes a filter 1100 coupled with an elongate element 1200, wherein a membrane 1400 extends along one or more portions of the filter 1100 and optionally along one or more portions of the elongate element 1200. Step S002 includes providing a delivery catheter. In various examples, the delivery catheter may be a COTS delivery catheter, consistent with the discussion above. In various examples, the delivery catheter therefore includes an elongate element having a distal end and a proximal end, and a lumen extending therethrough from the proximal end to the distal end. A COTS delivery catheter 6000 is shown in FIG. 6A, along with the embolic filter system 1000, including the filter 1100, the elongate element 1200, and the membrane 1400. The COTS delivery catheter 6000 may optionally include one or more connectors, such as connector 6100, which may include a hemostasis valve or other element. It should be appreciated that the embolic filter system 1000 is shown in FIG. 6A.

Turning back now to FIG. 5, at step S004, the proximal end of the embolic filter system 1000 is inserted into the lumen of the delivery catheter and proximally advanced through the lumen of the delivery catheter until the proximal end of the embolic filter system 1000 extends proximal to the proximal end of the delivery catheter. For example, as shown in FIG. 6B, the second end 1004 of the embolic filter system 1000 has been inserted into the lumen of the delivery catheter 6000 at the distal end 6002 of the delivery catheter 6000 and proximally advanced through the lumen of the delivery catheter 6000 until the second end 1004 of the embolic filter system 1000 extends proximal to the proximal end 6004 of the delivery catheter 6000.

Turning back now to FIG. 5, at step S006, the embolic filter system 1000 is proximally withdrawn until the filter 1100 is received within the lumen of the delivery catheter 6000. FIGS. 6C and 6D illustrate the proximal withdrawal of the embolic filter system 1000 relative to the delivery catheter 6000, where the embolic filter system 1000 is withdrawn such that the filter 1100 is partially received within the lumen of the delivery catheter 6000 in FIG. 6C, and where the embolic filter system 1000 is withdrawn such that the filter 1100 is completely received within the lumen of the delivery catheter 6000 in FIG. 6D.

With the filter 1100 completely received within the lumen of the delivery catheter 6000, as shown in FIG. 6D, the delivery catheter 6000 can be inserted into the vasculature of a patient and advanced to a treatment site therein, whereinafter the embolic filter system 1000 can be advanced relative to the delivery catheter 6000 (e.g., by one or more of distally advancing the embolic filter system 1000 relative to the delivery catheter 6000 and proximally withdrawing the delivery catheter 6000 relative to the embolic filter system 1000) such that the filter 1100 extends distally from the distal end 6002 of the delivery catheter 6000. In some examples, as mentioned above, one or more portions of the filter 1100, such as the capture section 1108, are configured to radially expand to interrupt blood flow to filter embolic debris therefrom.

For example, shown in FIGS. 6E and 6F is the embolic filter system 1000 being advanced distally relative to the delivery catheter 6000 such that the filter 1100 extends from the distal end 6002 of the delivery catheter 6000. FIG. 5E shows a portion of the filter 1100 extending from the distal end 6002 of the delivery catheter 6000, and partially deployed (e.g., radially expanded), and FIG. 6F shows the filter 1100 extending from the distal end 6002 of the delivery catheter 6000, fully deployed (radially expanded). It will be appreciated that FIGS. 6E and 6F are shown with the embolic filter system 1000 and delivery catheter 6000 outside of the body for clarity.

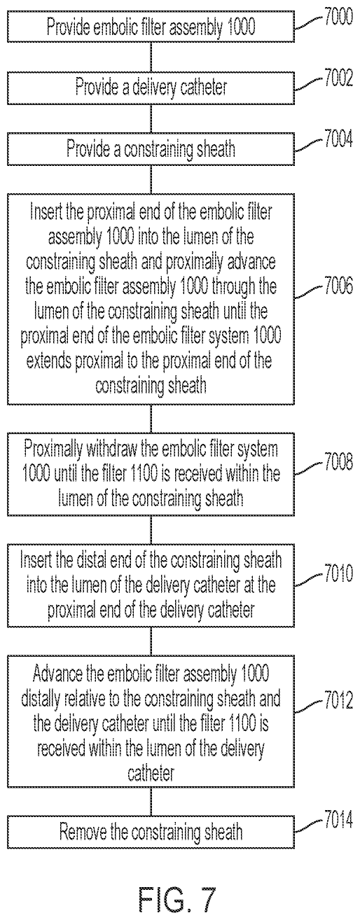

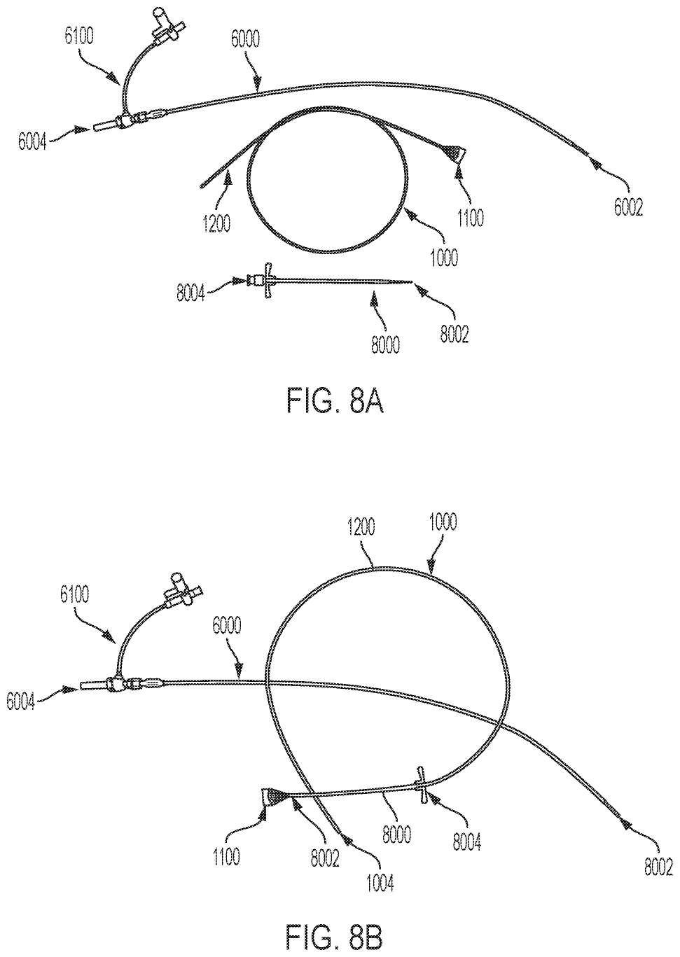

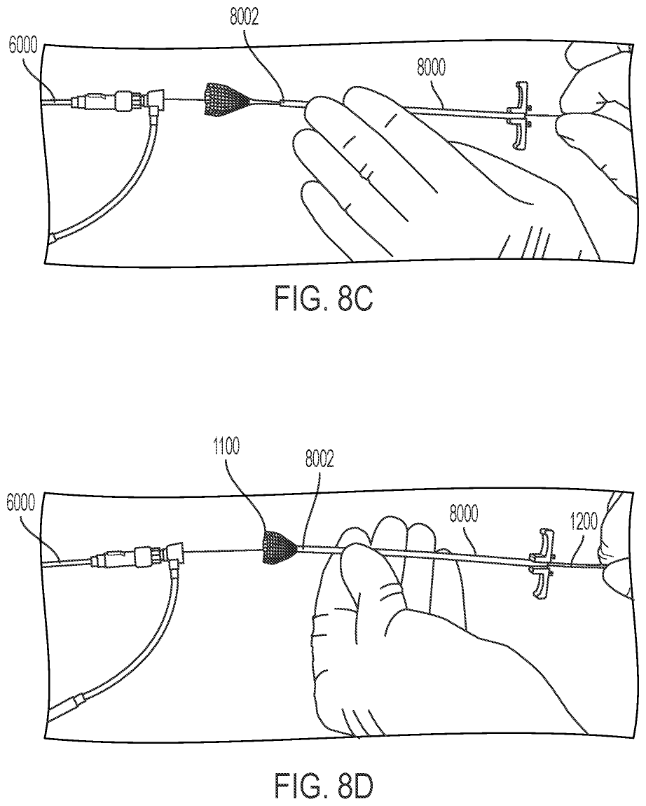

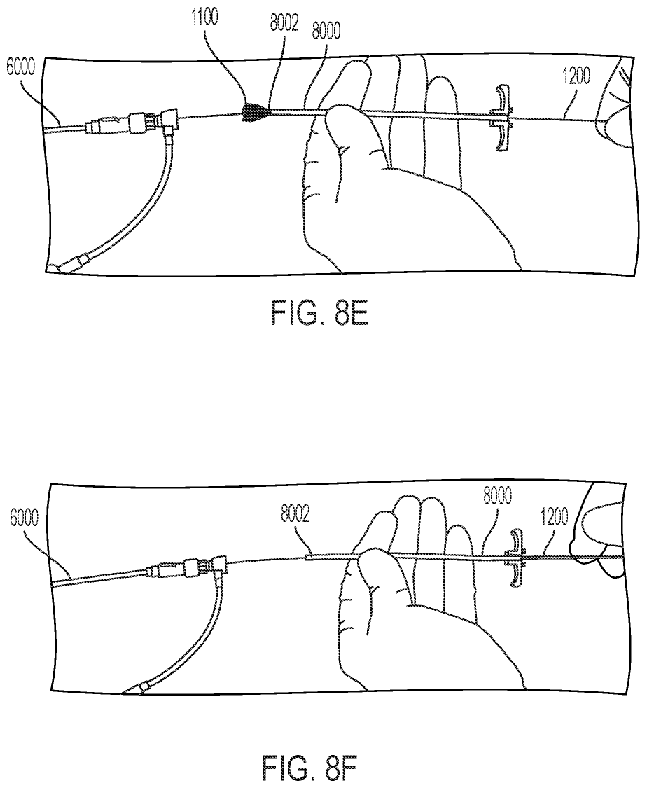

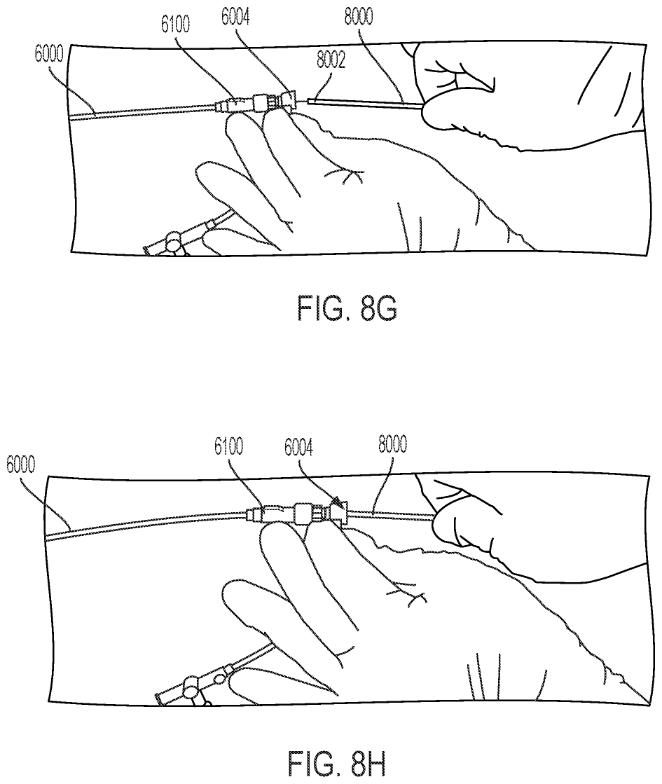

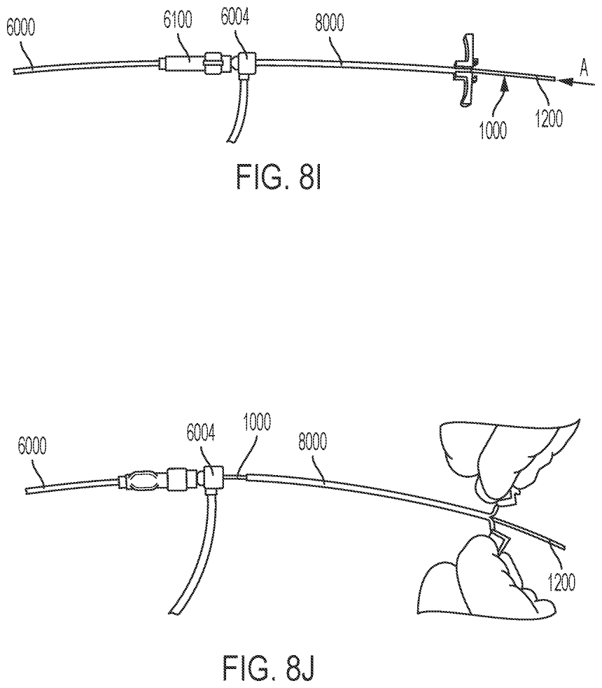

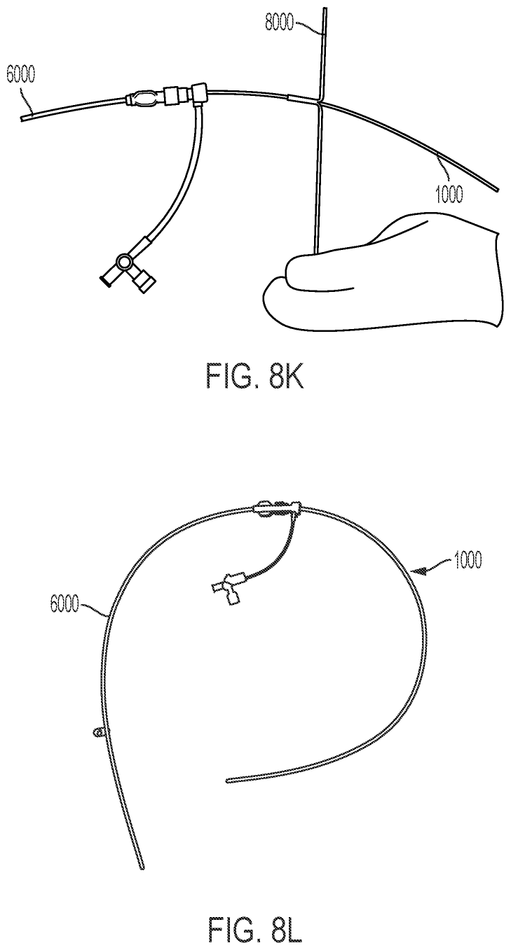

Turning now to FIG. 7 a flow chart is illustrated that outlines another example method for a medical device assembly including the embolic filter system 1000. As shown, step 7000 includes providing the embolic filter system 1000 as similarly discussed above with regard to step S000 of FIG. 5. Step 7002 includes providing a delivery catheter as similarly discussed above with regard to step S002 of FIG. 5. Step 7004 includes providing a constraining sheath, such as a COTS constraining sheath or a constraining sheath specifically designed for use in combination with the embolic filter system 1000. The constraining sheath may optionally be a constraining sheath that is splittable or that is otherwise configured to be torn-away from the embolic filter system 1000 and the delivery system. FIG. 8A provides an illustration of a delivery catheter 6000 with connector 6100, along with the embolic filter system 1000, and a constraining sheath 8000.

Turning back now to FIG. 7, at step 7006, the proximal end of the embolic filter system 1000 is inserted into the lumen of the constraining sheath and proximally advanced through the lumen of the constraining sheath 8000 until the proximal end of the embolic filter system 1000 extends proximal to the proximal end of the constraining sheath. For example, as shown in FIG. 8B, the second end 1004 of the embolic filter system 1000 has been inserted into the lumen of the constraining sheath 8000 at the distal end 8002 of the constraining sheath 8000 and proximally advanced through the lumen of the constraining sheath 8000 until the second end 1004 of the embolic filter system 1000 extends proximal to the proximal end 8004 of the constraining sheath 8000.

Turning back now to FIG. 7, at step 7008, the embolic filter system 1000 is proximally withdrawn until the filter 1100 is received within the lumen of the constraining sheath 8000. FIGS. 8C to 8F illustrate the proximal withdrawal of the embolic filter system 1000 relative to the constraining sheath 8000, where the embolic filter system 1000 is withdrawn such that the filter 1100 is partially received within the lumen of the constraining sheath 8000 in FIGS. 8C to 8E, and where the embolic filter system 1000 is withdrawn such that the filter 1100 is completely received within the lumen of the constraining sheath in FIG. 8F. In various examples, as described further below, the withdrawal of the embolic filter system 1000 relative to the constraining sheath 8000 may optionally be performed with the delivery catheter 6000 inserted within the vasculature. In some examples, the withdrawal of the embolic filter system 1000 relative to the constraining sheath 8000 may also optionally be performed with a guidewire extending through one or more of the delivery catheters 6000, the embolic filter system 1000, and the constraining sheath 8000, as shown in FIGS. 8C to 8F.

Additionally, in some examples, the elongate element 1200 may have one or more visible markers on the proximal end (e.g. the end of the elongate element 1200 that is being handled by the operator in FIG. 8D) and one or more visible markers on the distal end (e.g. proximate the filter 1100) such that the operator can see how far the filter 1100 coupled to the distal end of the elongate element 1200 is currently disposed within the patient's body by observing the position of each of the markers. In some examples, the proximal markers are visible to the unaided eye while the distal markers are visible under fluoroscopy (e.g., radiopaque). In some examples, one or both of the proximal and distal ends includes only one visible marker. In some example, the visible markers are located along a portion of the length of the elongate element 1200 in the regular or varying increments (e.g., increments of 1 mm, 0.5 cm, 1 cm, 2 cm, or any other suitable increments as deemed useful for the operator). Similarly, visible markers may also be located on the opposite end of the elongate element 1200 or along a length of the filter 1100 and/or the articulation section 1118. As mentioned, in some examples, the visible markers located on the distal end are radiopaque markers made from materials such as high-visibility tantalum or other metals or alloys that are visible in fluoroscopic images. By using the markers located on either or both the proximal and distal ends, the operator can better understand the relative position of the filter 1100 in the body of a patient.

Turning back now to FIG. 7, at step 7010, the distal end of the constraining sheath is inserted into the lumen of the delivery catheter at the proximal end of the delivery catheter. For example, turning now to FIGS. 8G and 8H, with the filter 1100 of the embolic filter system 1000 constrained within the lumen of the constraining sheath 8000, the distal end 8002 of the constraining sheath 8000 is inserted into the lumen of the delivery catheter 6000 at the proximal end 6004 of the delivery catheter 6000. In some example, this may include inserting the distal end 8002 of the constraining sheath 8000 into a connector of the delivery catheter 6000, such as connector 6100. FIG. 8G shows the constraining sheath 8000 with the filter 1100 of the embolic filter system 1000 constrained therein being advanced toward the proximal end 6004 of the delivery catheter 6000, and FIG. 8H shows the distal end 8002 of the constraining sheath 8000 inserted within the lumen of the delivery catheter 6000 at the proximal end 6004 of the delivery catheter 6000.

Turning back now to FIG. 7, at step 7012, with the distal end of the constraining sheath inserted in the lumen of the delivery catheter at the proximal end of the delivery catheter, the embolic filter system 1000 is distally advanced relative to the constraining sheath and the delivery catheter until the filter 1100 is received within the lumen of the delivery catheter. For example, as shown in FIG. 8I, with the distal end 8002 of the constraining sheath 8000 inserted in the lumen of the delivery catheter 6000 at the proximal end 6004 of the delivery catheter, the embolic filter system 1000 is distally advanced in the direction of arrow “A” relative to the constraining sheath 8000 and the delivery catheter 6000 until the filter 1100 is received within the lumen of the delivery catheter 6000. FIG. 8J shows, in part, the embolic filter system 1000 inserted into the lumen of the delivery catheter 6000 such that the filter 1100 is received within and constrained by the delivery catheter 6000 in a delivery configuration (e.g., radially constrained).

Turning back now to FIG. 7, after the filter 1100 of the embolic filter system 1000 is received within the lumen of the delivery catheter, the constraining sheath is removed in accordance with step 7014. In various examples, the constraining sheath 8000 is removed from the lumen of the delivery catheter 6000 during removal. In some examples, the constraining sheath 8000 proximally advanced along and relative to the elongate element 1200 of the embolic filter system 1000 until the distal end 8002 of the constraining sheath clears or translates to a position distal to the proximal end 1004 of the embolic filter system 1000. However, in some examples, as mentioned above, the constraining sheath 8000 is splittable or is otherwise configured to be torn away from the embolic filter system 1000 and the delivery catheter 6000. Such splittable constraining sheaths may provide ease of removal where one or more connectors (e.g., Tuohy-Borst connector) are coupled to the elongate element 1200 of the embolic filter system 1000 proximal to the constraining sheath 8000. In some such examples, the splittable constraining sheath can be removed from the embolic filter system 1000 and the delivery catheter 6000 without requiring removal of the connector coupled to the elongate element 1200 of the embolic filter system 1000 proximal to the constraining sheath 8000.

An example removal of such a splittable constraining sheath 8000 is shown in FIGS. 8J and 8K, where the constraining sheath 8000 is shown being split in to two sections for removal from the embolic filter system 1000 and the delivery catheter 6000. FIG. 8L shows the embolic filter system 1000 with the filter 1100 completely received within the lumen of the delivery catheter 6000.

With the filter 1100 completely received within the lumen of the delivery catheter 6000, as shown in FIG. 8L, the delivery catheter 6000 can be inserted into the vasculature of a patient and advanced to a treatment site therein, whereinafter the embolic filter system 1000 can be advanced relative to the delivery catheter 6000 (e.g., by one or more of distally advancing the embolic filter system 1000 relative to the delivery catheter 6000 and proximally withdrawing the delivery catheter 6000 relative to the embolic filter system 1000) such that the filter 1100 extends distally from the distal end 6002 of the delivery catheter 6000. As mentioned above, one or more portions of the filter 1100, such as the structural element of the capture section 1108, are configured to radially expand to interrupt blood flow to filter embolic debris therefrom.

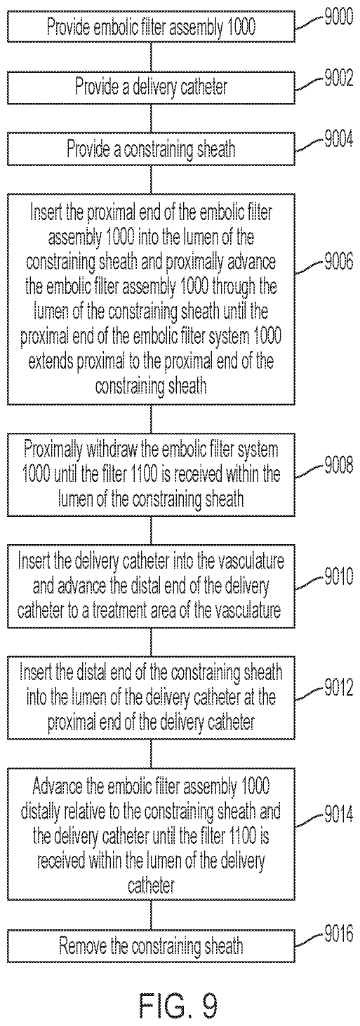

Turning now to FIG. 9 a flow chart is illustrated that outlines an example method for delivering a medical device including the embolic filter system 1000 to a region within a patient's vasculature. As shown, steps 9000 to 9008 are consistent with steps 7000 to 7008 described above with respect to FIG. 7. At step 9010, the delivery catheter is inserted into the vasculature of a patient and advanced until a distal end of the delivery catheter is positioned at a treatment area of the vasculature. Accordingly, it is to be appreciated that while the discussion above includes advancing the delivery catheter to a treatment area within a patient's vasculature after the embolic filter system 1000 is received within the delivery catheter 6000, in some examples, the delivery catheter 6000 may alternatively be inserted into the vasculature of the patient and advanced until a distal end of the delivery catheter is positioned at a treatment area of the vasculature prior to inserting the embolic filter system 1000 into the delivery catheter 6000.

At step 9012, the distal end of the constraining sheath is inserted into the lumen of the delivery catheter at the proximal end of the delivery catheter. This step is largely consistent with step 7010 of FIG. 7, with the exception that step 9012 is being performed with the delivery catheter in situ (i.e., while the delivery catheter is inserted within the patient's vasculature. Accordingly, reference is drawn to FIGS. 8G and 8H, which illustrate the distal end 8002 of the constraining sheath 8000 being inserted into the lumen of the delivery catheter 6000 at the proximal end 6004 of the delivery catheter 6000. Those of skill should thus appreciate that the inventive concepts of the present disclosure provide for the ability to perform the step of inserting the constraining sheath into the lumen of the delivery catheter at the proximal end of the delivery catheter in situ or alternatively prior to advancement of the delivery catheter to the treatment area within the vasculature.

Such a versatile system provides that the embolic filter system 1000 can be delivered to remote regions of the vasculature that might not be accessible with conventional systems. Such a system also provides that the embolic filter system 1000 can be delivered to remote regions of the vasculature while minimizing trauma to the vasculature. For instance, those of skill will appreciate that the stiffness of a delivery catheter increases as additional components are received within its lumen. Relatively stiff delivery catheters may not be operable to navigate tortuous anatomy to reach certain regions of the vasculature and/or may traumatize the vasculature as a result of inflexibility. The embolic filter system 1000 described herein provides that a relatively flexible delivery catheter can be first advanced to a treatment area within the vasculature (e.g., such as within or through a relatively tortuous region), without one or more additional components disposed therein that would otherwise operate to increase the stiffness of the delivery catheter. Moreover, such a configuration provides that the delivery catheter can operate as a protective boundary and bearing surface separating the embolic filter system 1000 from the surrounding vasculature as the embolic filter system 1000 is advanced to the treatment area.

Steps 9014 and 9016 are consistent with steps 7012 and 7014 described above with respect to FIG. 7. Similarly, as illustrated and described above, it is to be appreciated that after the filter 1100 of the embolic filter system 1000 is advanced through the lumen of the delivery catheter to the treatment site, the embolic filter system 1000 is operable to be deployed from the distal end of the delivery catheter (e.g., by one or more of distally advancing the embolic filter system 1000 relative to the delivery catheter and proximally withdrawing the delivery catheter relative to the embolic filter system 1000) such that the filter 1100 extends distally from the distal end of the delivery catheter and expands to interrupt blood flow to filter embolic debris therefrom.

The versatility of the embolic filter system 1000 illustrated and described herein also provides for ease of removal of the embolic filter system 1000 from the vasculature and repositioning of the same in-situ. For example, during or subsequent to a deployment of the embolic filter system 1000 within the vasculature, and operator can manipulate the angular relationship between the filter 1100 and the elongate element 1200 of the embolic filter system 1000 to achieve a better alignment of the filter 1100 with the vessel within which it is deployed. For instance, as mentioned above, the embolic filter system 1000 is operable to have a relative articulation occur between the filter 1100 and the elongate element 1200 by way of an articulation section 1118 bending or curving in response to advancement and retraction of the elongate element 1200. When the filter 1100 is deployed within a vessel, one or more portions of the filter engage the vessel wall, thereby creating an engagement between the filter 1100 and the vessel.

With the filter 1100 engaged with the vessel, the elongate element 1200 is operable to be advanced or retracted. Under certain conditions, advancement of the elongate element 1200 with the filter 1100 engaged, at least in part, with the vessel wall causes the embolic filter system 1000 to undergo a compressive loading condition. In certain instances, such as those where the filter 1100 is improperly aligned with the vessel in which it is deployed, such a compressive loading condition causes the articulation section 1118 of the embolic filter system 1000 to bend, thereby causing a relative articulation between the filter 1100 (or at least a distal end thereof) and the elongate element 1200, as discussed above. Conversely, under certain conditions, retraction of the elongate element 1200 with the filter 1100 engaged, at least in part, with the vessel wall causes the embolic filter system 1000 to undergo a tensile loading condition. In certain instances, such as those where the filter 1100 misaligned with the elongate element 1200, such a tensile loading condition causes the articulation section 1118 of the embolic filter system 1000 to straighten, thereby causing a relative articulation between the filter 1100 (or at least a distal end thereof) and the elongate element 1200 such that the filter 1100 and the elongate element 1200 migrate toward alignment with one another. Thus, the elongate element 1200 can be advanced and retracted to cause articulation between the filter 1100 (or at least a distal end thereof) and the elongate element 1200, that can be utilized to achieve a proper alignment of the filter 1100 within the vessel. It should be appreciated that proper alignment of the filter 1100 within the vessel does not require alignment between the filter 1100 and the elongate element 1200, and may require misalignment between the filter 1100 and the elongate element 1200.

While the embolic filter system 1000 of the various examples and illustrations described above includes a filter 1100 having an articulation section 1118 incorporated therein, in some alternative examples, the embolic filter system 1000 may additionally or alternatively include one or more independent articulation elements that are positioned proximal to the filter 1100 and that provide for articulation between the filter 1100 and one or more portions of the elongate element 1200. That is, in some example, the embolic filter system 1000 includes an articulation element that is independent of (e.g., not part of) the filter 1100. For instance, the filter 1100 may include the structural element of the capture section 1108 without also including the articulation section 1118.

The articulation element in such examples may be consistent in form in function with the articulation section 1118 of the filter 1100 described above, with the exception that the articulation element is not an integral portion of the filter 1100 but is instead an independent component that is coupled (either directly or indirectly) to one or more of the filter 1100 and the elongate element 1200. Thus, in some examples, the articulation element includes a tubular construct that has been helically cut or slotted. As mentioned above, in those examples including a cut tube, the cuts in the tube to form the coil/helix or slotted segment extend through the thickness of the tube (e.g., from an exterior surface of the tube to the interior surface of the tube) such that the interior lumen of the tube is exposed. Such full thickness cuts in the tube provide gaps that can accommodate bending in one or more related portions of the tube (e.g., bending of one or more helical windings).

FIGS. 10A-10C shows exemplary flexion elements 1300. As previously disclosed, the flexion elements 1300 may include a variety of configurations including serpentine-shaped (e.g., FIG. 10A), coil-shaped (e.g., FIG. 10B), and zig zag-shaped (e.g., 10C). Furthermore, in various embodiments, the flexion elements 1300 may be formed continuously with the capture section 1108 (e.g., the plurality of strut elements 1110), wherein the flexion elements 1300 are thinned, narrowed, or otherwise have a reduced profile that facilitates increased flexion of the flexion elements 1300 relative to the other portions of the capture section 1108.

In some embodiments, the elongate element 1200 is configured such that its length can be easily modified in association with and endovascular procedure. For instance, in some examples, the elongate element 1200 is operable to be cut such that a length of the elongate element can be modified from a first length, to a second shorter length. In some examples, the elongate element 1200 is configured such that the length of elongate element 1200 can be modified while the embolic filter system 1000 is received within the lumen of the delivery catheter 6000. In some examples, an attachable/detachable hub is coupled to the proximal end of the elongate element 1200 to fluidly seal the lumen of the delivery catheter 6000. For example, the hub may have a Luer taper connection, a hose barb connection, or a combination thereof (e.g., Luer-to-barb fitting connection) as used to form a leak-free connection at the proximal end of the elongate element 1200, as suitable. In some examples, the hub may be permanently attached or coupled to the proximal end of the elongate element 1200 and in others the hub may be removably attached thereto.

In some examples, the elongate element 1200 includes a plurality of predetermined sections that are configured to be removed. For instance, in some examples, the elongate element 1200 includes a first removable section and a second removable section, such that either one or both of the first and second removable sections can be removed to modify the length of the elongate element from the first length to the second shorter length. In some examples, the removable sections may be configured to be removed by way of cutting. In some other examples, the removable sections may be configured to be additionally or alternatively removed by way of twisting, bending, or pulling the removable section relative to the remainder of the elongate element 1200.

In some examples, one or more portions or components of the embolic filter system 1000, such as the elongate element 1200, may be color-coded to indicate a diameter of the elongate element 1200, wherein a first color indicates a first diameter (e.g., 6 Fr) and wherein a second color indicates a second different diameter. Such color-coding can help users identify a proper diameter for used with a COTS delivery catheter in association with an endovascular procedure.

It should be appreciated that the configurations discussed herein are scalable in that they can be scaled up or scaled down for different applications. That is, while certain of the configurations discussed herein are illustrated and described in association with placement within the aortic arch, for example, the versatility of the system provides for implementation in virtually any other area of the patient's vasculature. For example, the various configurations discussed herein may be scaled for application within various peripheral vessels and lumens such as the brachiocephalic artery, and/or the carotid artery, and/or the subclavian artery. Likewise, as it relates to the aortic arch, the present disclosure can be used in connection with femoral, transapical and thoracotomy approaches. Moreover, this disclosure should not be interpreted as limiting application to the vessels proximate the heart. For instance, the devices and systems described herein may be implemented throughout the vasculature of the body including vasculature above and below the heart to prevent the migration of embolic debris during various other revascularization procedures. Additionally, the embodiments can be used in connection with not just humans, but also various organisms having mammalian anatomies. Thus, it is intended that the embodiments described herein cover the modifications and variations within the scope of this disclosure. As such, the embolic filter system 1000 may be formed in a variety of different sizes, which may optionally be based on COTS delivery catheter sizes such that the embolic filter system 1000 can be produced in a variety of sizes that can be used in association with the variety of sized of COTS delivery catheters. As mentioned above, one or more components of the embolic filter system 1000 may be color coded based on such sizing.

The inventive scope of this application has been described above both generically and with regard to specific examples. It will be apparent to those skilled in the art that various modifications and variations can be made in the examples without departing from the scope of the disclosure. Likewise, the various components discussed in the examples discussed herein are combinable. Thus, it is intended that the examples cover the modifications and variations of the inventive scope.

1. A medical device comprising:
an elongate element having a first end and a second end; and
an embolic filter assembly comprising a frame having a capture section distal to the attachment section, the capture section including a plurality of strut elements arranged to define a plurality of cells having cell ends and at least one flexion element operably coupled to and between cell ends of two adjacent cells of the plurality of cells such that the frame is operable to bend between the cell ends.

2. The medical device of claim 1, wherein the capture section is radially expandable relative to the attachment section such that the embolic filter assembly is configured to transition from a compressed state toward an expanded state in situ.

3. The medical device of claim 1, wherein the capture section includes a seal establishment zone operable to establish a seal with tissue of a patient and a flexion zone operable to flex to conform to tortious anatomy of a patient.

4. The medical device of claim 3, wherein the flexion element defines the flexion zone in which the frame has greater flexibility than in adjacent portions of the frame.

5. The medical device of claim 1, wherein the flexion element is serpentine shaped having at least one curve in a first direction and at least one curve in a second, opposite direction from the first direction.

6. The medical device of claim 1, wherein the plurality of cells are closed cells.

7. The medical device of claim 6, wherein each of the plurality of closed cells includes a first apex and a second apex opposite the first apex.

8. The medical device of claim 7, wherein the at least one flexion element is operably coupled between the proximal apex of a first closed cell of the plurality of closed cells and the distal apex of a second closed cell of the plurality of closed cells.

9. The medical device of claim 8, wherein the plurality of closed cells form a first cell row and a second cell row along a longitudinal length of the frame, wherein each distal apex of the plurality of closed cells of the first cell row is operably coupled to each proximal apex of the plurality of closed cells of the second cells via the flexion element.

10. The medical device of claim 1, wherein the at least one flexion element is adapted to bend at a plurality of flex points between first and second ends of the flexion element.

11. The medical device of claim 1, wherein the at least one flexion element is adapted to extend in length.

12. An embolic filter assembly for deployment in a lumen of a patient, the embolic filter comprising:
a frame disposed about a longitudinal axis and operable to expand from a smaller, collapsed configuration to a larger, expanded configuration, the frame including a plurality of frame elements defining a sealing section and a capture section, the sealing section configured to interface with the lumen of the patient, the capture section including flexion elements positioned between frame elements within the capture zone, the frame being operable to bend away from the longitudinal axis at the flexion elements more than adjacent portions of the frame without flexion elements.

13. The embolic filter of claim 12, wherein the flexion elements are aligned around a circumference of the frame at a common, longitudinal position along a length of the frame.

14. The embolic filter of claim 12, wherein the frame is operable to be partially deployed such that the sealing section is operable to expanded to an expanded diameter while at least a portion of the capture section is maintained at a collapsed diameter.

15. The embolic filter of claim 14, wherein the capture section is operable to filter fluid flowing through the capture section when the capture section is partially deployed.

16. The embolic filter of claim 12, wherein the frame is operable to angulate at the flexion elements to maintain the frame in an orthogonal orientation within the lumen when deployed.

17. The embolic filter of claim 16, wherein the flexion elements include a curved shape, a bent shape, a zigzag shape, a serpentine shape, or a coil shape.

18. The embolic filter of claim 12, wherein the frame elements form a plurality of closed cells, each closed cell including a proximal apex and a distal apex.

19. The embolic filter of claim 18, wherein a first flexion element is operably coupled between the proximal apex of a first closed cell of the plurality of closed cells and the distal apex of a second closed cell of the plurality of closed cells.

20. An embolic filter assembly for deployment in a lumen of a patient, the embolic filter comprising:
a frame disposed about a longitudinal axis and operable to expand from a smaller, collapsed configuration to a larger, expanded configuration, the frame including a plurality of frame elements defining a sealing section and a capture section, the sealing section configured to interface with the lumen of the patient, the capture section including flexion elements positioned between frame elements within the capture zone, the frame being operable to be partially deployed such that the sealing section is operable to expanded to an expanded diameter while at least a portion of the capture section is maintained at a collapsed diameter.
